(19) United States
(12) Patent Application Publication
(10) Pub. No.: US 2026/0103502 A1
(43) Pub. Date: Apr. 16, 2026

(54) Chimeric Antigen Receptor and Natural Killer Cells Expressing Same

(71) Applicant: GC Cell Corporation, Yongin-si, Gyeonggi-do (KR)

(72) Inventors: Yu Kyeong Hwang, Yongin-si, Gyeonggi-do (KR); Sung Yoo Cho, Yongin-si, Gyeonggi-do (KR); Sung Yong Won, Yongin-si, Gyeonggi-do (KR); Ho Yong Lim, Yongin-si, Gyeonggi-do (KR); Jung Hyun Her, Yongin-si, Gyeonggi-do (KR); Mi Young Jung, Yongin-si, Gyeonggi-do (KR); Hyun Ah Kim, Yongin-si, Gyeonggi-do (KR); Su Hyun Gwon, Yongin-si, Gyeonggi-do (KR); Eun Sol Lee, Yongin-si, Gyeonggi-do (KR); Han Sol Kim, Yongin-si, Gyeonggi-do (KR)

(21) Appl. No.: 19/322,080

(22) Filed: Sep. 8, 2025

Related U.S. Application Data
(63) Continuation of application No. 17/845,793, filed on Jun. 21, 2022, now Pat. No. 12,435,118.
Application No. 17/845,793 is a continuation of application No. 16/474,426, now abandoned.

(30) Foreign Application Priority Data
Dec. 28, 2016 (KR) 1020160181119

(51) Int. Cl. C07K 14/725; A61K 40/15; A61K 40/31; A61K 40/42; C07K 16/28; C12N 5/0783

(52) U.S. Cl. CPC C07K 14/7051; A61K 40/15; A61K 40/31; A61K 40/4221; A61K 40/4224; C07K 16/2815; C07K 16/2818; C07K 16/283; C07K 16/2851; C12N 5/0646; A61K 2239/22; A61K 2239/48; A61K 2239/49; A61K 2239/55; C07K 2317/622; C07K 2317/73; C07K 2319/03; C07K 2319/33

(57) ABSTRACT

The present invention provides a chimeric antigen receptor and natural killer cells expressing the same, and particularly, a chimeric antigen receptor (CAR) which includes an intracellular signaling domain including the whole or a portion of an OX40 ligand (CD252), thereby having excellent effects of increasing anticancer activity of immune cells, and immune cells expressing the same.

### CROSS REFERENCE TO RELATED APPLICATIONS

This application is a continuation of U.S. application Ser. No. 17/845,793, filed on Jun. 21, 2022, which is a continuation of U.S. application Ser. No. 16/474,426 filed Jun. 27, 2019, which is a National Stage of International Application No. PCT/KR2017/015635 filed Dec. 28, 2017, claiming priority based on Korean Patent Application No. 10-2016-0181119 filed Dec. 28, 2016.

### SEQUENCE LISTING

This application contains a Sequence Listing that has been submitted electronically as an XML file named “49755-0008003_SL_ST26.XML.” The XML file, created on Sep. 3, 2025, is 140,960 bytes in size. The material in the XML file is hereby incorporated by reference in its entirety.

### TECHNICAL FIELD

The present invention relates to a chimeric antigen receptor and natural killer cells expressing the same.

### BACKGROUND ART

Human natural killer cells (NK cells) play an important role in innate immune defense against malignant lymphoma cells, and thus may be suitable for adoptive immune therapy (i.e., adoptive cellular immunotherapy). However, due to difficulties in ex vivo cell expansion and differences in activities of NK cells in individual patients, it is difficult to use the NK cells.

In a field of immune cell therapy using T cells, that is, T cell therapy field, a chimeric antigen receptor for treatment of autoimmune cells has been developed. The chimeric antigen receptor has been known to help activation of T cells by reprogramming T cells in order to improve treatment effects to specific cancers or overcome resistance against T cell treatment.

OX40 ligand (CD252) is a protein belonging to a TNFR superfamily, and is known to be expressed in antigen-presenting cells (APCs), some of NK cells and some of B cells, as well as known to be expressed in several hours to a few days after activation of these cells.

As a receptor of OX40 ligand (CD252), OX40 (CD134) is known to be expressed in T cells, and also known to be expressed in 24 hours after activation of T cells by antigen and CD28. It is known that expression of CD134 may further enhance a response of T cells by CD28 activation, so as to increase proliferation of T cells, and cytokine secretion and survival.

However, a role of OX40 ligand in enhancement of anticancer activity of NK cells is not well known, and an attempt to use the OX40 ligand for the chimeric antigen receptor is still not yet reported.

In order to research functional roles of CD252 in antitumor response of NK cells, the present inventors have constructed two different kinds of NK receptors containing CAR. First, a variety of stimulatory cofactors known to increase cytotoxicity of NK cells in a signaling domain were genetically engineered to produce lentivirus encoding high affinity recombination FCRG3A V158 variants (CD16V). CD16V receptors including different intercellular signaling molecules were transduced in NK cells, followed by detecting expression thereof. Further, antitumor responses of the transduced NK cells were investigated using lymphoma cell lines in vitro in the presence of rituximab specific to CD20 molecules.

As a second approach, functional roles of CD252 in NKG2D-mediated antitumor activity was tested. NKG2D as a key receptor in NK cell activation can recognize MHC class 1 chain-related A (MICA), MICB and other diverse UL 16-binding proteins (ULBP), which are preferentially expressed after genotoxic stress, infection and/or cytotoxicity.

A variety of NKG2D chimeric antigen receptors containing different signaling molecules were genetically engineered, transduced, and expressed in NK92MI cells. Thereafter, the transduced cells were subjected to evaluation of antitumor effects in vitro.

The present inventors have found that, as one approach to overcome limitations of current immune cell therapy like NK cells, a chimeric antigen receptor containing OX40 ligand has significantly improved in anticancer activities of NK cells, as compared to the other signaling domains known, thereby completing the present invention.

### DISCLOSURE

### Technical Problem

Accordingly, it is an object of the present invention to provide a chimeric antigen receptor capable of increasing anticancer immune cell treatment efficiency using natural killer (NK) cells.

### Technical Solution

1. A chimeric antigen receptor, including: an intracellular signaling domain which includes the whole or a portion of OX40 ligand (CD252).
2. The chimeric antigen receptor according to the above 1, further including: a transmembrane domain linked to the intracellular signaling domain; a spacer domain linked to the transmembrane domain; and an extracellular domain linked to the spacer domain.
3. The chimeric antigen receptor according to the above 2, further comprising a signal sequence linked to the extracellular domain.
4. The chimeric antigen receptor according to the above 3, wherein the signal sequence includes the whole or a portion of CD16 and the signal sequence includes the whole or a portion of CD8a, respectively.
5. The chimeric antigen receptor according to the above 2, wherein the extracellular domain includes the whole or a portion of any one selected from the group consisting of an antigen-binding fragment of an antibody, an Fc receptor, a natural cytotoxicity receptor, NKG2D, 2B4 and DNAM-1.
6. The chimeric antigen receptor according to the above 5, wherein the antigen-binding fragment is Fab fragment, F(ab′) fragment, F(ab′)2 fragment or Fv fragment.
7. The chimeric antigen receptor according to the above 6, wherein the Fv fragment is a single-chain variable fragment (ScFv).
8. The chimeric antigen receptor according to the above 5, wherein the Fc receptor is selected from the group consisting of CD16, CD32, CD64, CD23, CD89 and variants thereof.
9. The chimeric antigen receptor according to the above 8, wherein the Fc receptor is CD16 or variants thereof.
10. The chimeric antigen receptor according to the above 5, wherein the natural cytotoxicity receptor is selected from the group consisting of NKp46, NKp30, NKp44, NKp80 and NKp65 receptors.
11. The chimeric antigen receptor according to the above 2, wherein the spacer domain includes the whole or a portion of any one selected from the group consisting of CD8α and CD28.
12. The chimeric antigen receptor according to the above 2, wherein the transmembrane domain includes the whole or a portion of any one selected from the group consisting of CD8α and CD28.
13. The chimeric antigen receptor according to the above 1, wherein the intracellular signaling domain further includes the whole or a portion of CD3-zeta.
14. The chimeric antigen receptor according to the above 13, wherein the whole or a portion of CD3-zeta and the whole or a portion of OX40 ligand are arranged in order from a cell membrane toward an inside of the cell.
15. The chimeric antigen receptor according to the above 2, further comprising an AAA linker between the extracellular domain and the spacer domain.
16. A chimeric antigen receptor, comprising intracellular signaling domains which include: a first intracellular signaling domain including the whole or a portion of any one selected from the group consisting of CD28 and 4-1BB; a second intracellular signaling domain including the whole or a portion of any one selected from the group consisting of OX40 ligand, OX40 and 4-1BB; and a third intracellular signaling domain including the whole or a portion of CD3-zeta, wherein the first, second and third intracellular signaling domains are arranged in order from a cell membrane toward an inside of the cell.
17. The chimeric antigen receptor according to the above 16, further including: a transmembrane domain linked to the intracellular signaling domain; a spacer domain linked to the transmembrane domain; and an extracellular domain linked to the spacer domain.
18. The chimeric antigen receptor according to the above 17, further comprising a signal sequence linked to the extracellular domain.
19. The chimeric antigen receptor according to the above 18, wherein the signal sequence includes the whole or a portion of CD16 and the signal sequence includes the whole or a portion of CD8a, respectively.
20. The chimeric antigen receptor according to the above 17, wherein the extracellular domain includes the whole or a portion of any one selected from the group consisting of an antigen-binding fragment of an antibody, an Fc receptor, a natural cytotoxicity receptor, NKG2D, 2B4 and DNAM-1.
21. The chimeric antigen receptor according to the above 17, wherein the spacer domain includes the whole or a portion of any one selected from the group consisting of CD8α and CD28.
22. The chimeric antigen receptor according to the above 17, wherein the transmembrane domain includes the whole or a portion of any one selected from the group consisting of CD8α and CD28.
23. The chimeric antigen receptor according to the above 16, wherein the first intracellular signaling domain includes the whole or a portion of CD28; the second intracellular signaling domain includes the whole or a portion of the OX40 ligand; and the third intracellular signaling domain includes the whole or a portion of CD3-zeta.
24. The chimeric antigen receptor according to the above 17, further comprising an AAA linker between the extracellular domain and the spacer domain. 25. An immune cell expressing the chimeric antigen receptor according to any one of the above 1 to 24.
26. The immune cell according to the above 25, wherein the immune cell is a natural killer cell (NK cell).
27. A pharmaceutical composition for treatment of tumor, comprising the immune cell according to the above 25 as an active ingredient.
28. The pharmaceutical composition according to the above 27, further comprising an antibody as an active ingredient when the extracellular domain is an Fc receptor.
29. A nucleic acid sequence encoding the chimeric antigen receptor according to any one of the above 1 to 24.
30. The nucleic acid sequence according to the above 29, wherein the nucleic acid sequence encodes one or more amino acid sequences selected from the group consisting of SEQ ID NOs: 33, 41, 43, 45, 47, 49, 51, 53, 55, 69, 71, 77, 81, 83, 85, 87, 89, 91 and 93, or variants thereof having a sequence identity of 80% or more.
31. The nucleic acid sequence according to the above 30, wherein the nucleic acid sequence includes one or more nucleic acid sequence selected from the group consisting of SEQ ID NOs: 32, 40, 42, 44, 46, 48, 50, 52, 54, 68, 70, 76, 80, 82, 84, 86, 88, 90 and 92, or variants thereof having a sequence identity of 80% or more.
32. A method for treatment of tumor, comprising administering the immune cell according to the above 25 to a subject.

### Advantageous Effects

The chimeric antigen receptor according to the present invention is excellent in NK cell activation efficiency.

The chimeric antigen receptor according to the present invention may be used along with various cancer-target antibodies depending on cancer types to be targeted.

The chimeric antigen receptor according to the present invention is applicable to diverse cancer types by applying different antigen recognition sites.

The NK cells expressing the chimeric antigen receptor according to the present invention are excellent in cytotoxicity on cancer cells.

The NK cells expressing the chimeric antigen receptor according to the present invention may be usefully used in immune cell treatment.

### DESCRIPTION OF DRAWINGS

FIG. 1A shows CAR expression levels of NK92MI cells transduced with CD16V-Z CAR (1^{st} generation), CD16V-28Z CAR (2^{nd} generation), CD16V-BBZ CAR (2^{nd} generation), CD16V-OX40Z CAR (2^{nd} generation) or CD16V-28OX40LZ CAR (3^{rd} generation), respectively, according to an embodiment of the present invention.

FIG. 1B shows intrinsic cell killing ability (i.e., ‘cytotoxicity’) of NK92MI cells transduced with CD16V-Z CAR (1^{st} generation), CD16V-28Z CAR (2^{nd} generation), CD16V-BBZ CAR (2^{nd} generation), CD16V-OX40Z CAR (2^{nd} generation) or CD16V-28OX40LZ CAR (3^{rd} generation), respectively, which is obtained through assessment of cytotoxicity on K562 cells.

FIG. 1C shows assessment of NK cell-mediated cytotoxicity of NK92MI cells transduced with CD16V-Z CAR (1^{st} generation), CD16V-28Z CAR (2^{nd} generation), CD16V-BBZ CAR (2^{nd} generation), CD16V-OX40Z CAR (2^{nd} generation) or CD16V-28OX40LZ CAR (3^{rd} generation), respectively, according to an embodiment of the present invention, when combined with an antibody to Ramos cells.

FIG. 1D shows CAR expression levels of NK92MI cells transduced with CD16V-Z CAR (1^{st} generation) and CD16V-OX40LZ CAR (2^{nd} generation), respectively, according to an embodiment of the present invention.

FIG. 1E shows assessment of NK cell-mediated cytotoxicity of NK92MI cells transduced with CD16V-Z CAR (1^{st} generation) and CD16V-OX40LZ CAR (2^{nd} generation), respectively, according to an embodiment of the present invention, when combined with an antibody to Ramos cells.

FIG. 1F shows CAR expression levels of NK92MI cells transduced with CD16V-Z CAR (1^{st} generation) and CD16V-ZOX40L CAR (2^{nd} generation), respectively, according to an embodiment of the present invention.

FIG. 1G shows assessment of NK cell-mediated cytotoxicity of NK92MI cells transduced with CD16V-Z CAR (1^{st} generation) or CD16V-ZOX40L CAR (2^{nd} generation), respectively, according to an embodiment of the present invention, when combined with an antibody to Ramos cells.

FIG. 2A shows CAR expression levels of NK92MI cells transduced with CD16V-Z CAR (1^{st} generation), CD16V-BBZ CAR (2^{nd} generation) or CD16V-BBOX40LZ CAR (3^{rd} generation), respectively, according to an embodiment of the present invention.

FIG. 2B shows assessment of NK cell-mediated cytotoxicity of NK92MI cells expressing CD16V-Z CAR (1^{st} generation), CD16V-BBZ CAR (2^{nd} generation) or CD16V-BBOX40LZ CAR (3^{rd} generation), respectively, according to an embodiment of the present invention, when combined with an antibody to Ramos cells.

FIG. 3A shows CAR expression levels of NK92MI cells transduced with CD16V-28OX40LZ CAR, CD16V-28OX40Z CAR or CD16V-28BBZ CAR, respectively, all of which are 3^{rd} generation CARs, according to an embodiment of the present invention.

FIG. 3B shows assessment of NK cell-mediated cytotoxicity of NK92MI cells expressing CD16V-28OX40LZ CAR, CD16V-28OX40Z CAR or CD16V-28BBZ CAR, respectively, all of which are 3^{rd} generation CARs, according to an embodiment of the present, when combined with an antibody to Ramos cells.

FIG. 4A shows CAR expression levels of NK92MI cells transduced with CD16V-28(H)BBZ CAR, CD16V-28(H)OX40Z CAR, or CD16V-28(H)OX40LZ CAR, respectively, all of which are 3^{rd} generation CARs each containing CD28 at a hinge, according to an embodiment of the present invention.

FIG. 4B shows assessment of NK cell-mediated cytotoxicity of NK91MI cells expressing CD16V-28(H)BBZ CAR, CD16V-28(H)OX40Z CAR or CD16V-28(H)OX40LZ CAR, respectively, all of which are 3^{rd} generation CARs each having CD28 at a hinge, according to an embodiment of the present invention, when combined with an antibody to Ramos cells.

FIG. 5A shows CAR expression levels of NK92MI cells transduced with NKG2D-Z CAR (1^{st} generation), NKG2D-28Z CAR (2^{nd} generation), NKG2D-BBZ CAR (2^{nd} generation) or NKG2D-OX40Z CAR (2^{nd} generation), respectively, according to an embodiment of the present invention.

FIG. 5B shows assessment of NK cell-mediated cytotoxicity of NK92MI cells transduced with NKG2D-Z CAR (1^{st} generation), NKG2D-28Z CAR (2^{nd} generation), NKG2D-BBZ CAR (2^{nd} generation) or NKG2D-OX40Z CAR (2^{nd} generation), respectively, against human breast cancer cell lines MCF-7, according to an embodiment of the present invention.

FIG. 6A shows CAR expression levels of NK92MI cells transduced with NKG2D-Z CAR (1^{st} generation), NKG2D-28BBZ CAR (3^{rd} generation) including CD28 signaling domain or NKG2D-28OX40Z CAR (3^{rd} generation), respectively, according to the present invention.

FIG. 6B shows assessment of NK cell-mediated cytotoxicity of NK92MI cells transduced with NKG2D-Z CAR (1^{st} generation), NKG2D-28BBZ CAR including CD28 signaling domain (3^{rd} generation) or NKG2D-28OX40Z CAR (3^{rd} generation), respectively, against human breast cancer cell lines MCF-7, according to an embodiment of the present invention.

FIG. 7A shows CAR expression levels of NK92MI cells transduced with NKG2D-Z CAR (1^{st} generation), NKG2D-28Z CAR (2^{nd} generation), NKG2D-28(H)OX40LZ CAR (3^{rd} generation) or NKG2D-AAA-28(H)OX40LZ CAR (3^{rd} generation) including AAA sequence between NKG2D extracellular domain and CD28 hinge, respectively, according to an embodiment of the present invention.

FIG. 7B shows assessment of NK cell-mediated cytotoxicity of NK92MI cells transduced with NKG2D-Z CAR (1^{st} generation), NKG2D-28Z CAR (2^{nd} generation), NKG2D-28(H)OX40LZ CAR (3^{rd} generation) or NKG2D-AAA-28(H)OX40LZ CAR (3^{rd} generation) including AAA sequence between NKG2D extracellular domain and CD28 hinge, respectively, against human breast cell lines MCF-7, according to an embodiment of the present invention.

FIG. 8A shows CAR expression levels of NK92MI cells transduced with NKG2D-Z CAR (1^{st} generation) or NKG2D-AAA-28(H)OX40LZ CAR (3^{rd} generation) including AAA sequence between NKG2D extracellular domain and CD28 hinge, respectively, according to an embodiment of the present invention.

FIG. 8B shows expression levels of different NKG2D ligands in human lung cancer cell lines H1299 and H1944 according to an embodiment of the present invention.

FIG. 8C shows assessment of NK-mediated cytotoxicity of NK92MI cells transduced with NKG2D-Z CAR (1^{st} generation) or NKG2D-AAA-28(H)OX40LZ (3^{rd} generation) including AAA sequence between NKG2D extracellular domain and CD28 hinge, respectively, against human lung cancer cell lines H1299 and H1944, according to an embodiment of the present invention.

### BEST MODE

The present invention discloses a chimeric antigen receptor and NK cells expressing the same, and more particularly, a chimeric antigen receptor (CAR) with excellent effects of increasing anticancer activity of immune cells, by including an intracellular signaling domain which contains the whole or a portion of OX40 ligand (CD252), as well as immune cells expressing the same.

Hereinafter, the present invention will be described in detail.

The chimeric antigen receptor of the present invention may include an intracellular signaling domain which contains the whole or a portion of OX40 ligand (CD252).

According to one embodiment of the present invention, the chimeric antigen receptor may be one further including a transmembrane domain linked to the intracellular signaling domain; a spacer domain linked to the transmembrane domain; and an extracellular domain linked to the spacer domain. In addition, according to one embodiment of the present invention, the chimeric antigen receptor may be one further including a signal sequence linked to a terminal of the extracellular domain that is not linked with the spacer domain. According to one embodiment of the present invention, the above-described domains may be directly linked to one another or may be linked by a linker.

According to one embodiment of the present invention, the signal sequence may allow the extracellular domain to be positioned outside a cell membrane of immune cells (e.g., NK cells) when the chimeric antigen receptor is expressed. For example, the signal sequence may include the whole or a portion of CD16.

According to one embodiment of the present invention, the extracellular domain is a domain to be specifically linked to an antibody or to specifically recognize an antigen, and may include, for example, an Fc receptor, an antigen-binding fragment such as a single-chain variable fragment (ScFv) in an antibody, a natural cytotoxicity receptor, NKG2D, 2B4 or DNAM-1. Accordingly, in the present disclosure, the term “extracellular domain” is used with the same meanings as “antigenic recognition site,” “antigen-binding fragment” and/or “antibody binding site.”

The chimeric antigen receptor according to one embodiment of the present invention may include an Fc receptor as the extracellular domain and thus be used associated with different antibodies depending on cell types of cancer to be treated. According to one embodiment, the Fc receptor may be any one selected from the group consisting of CD16, CD32, CD64, CD23 and CD89. According to a more specific embodiment, the Fc receptor may be one including the whole or a portion of the CD16 V158 variant (CD16V).

According to another embodiment, the chimeric antigen receptor of the present invention may further include an antigen-binding fragment of an antibody, as an extracellular domain, which can directly recognize the antigen without co-administration along with the antibody. According to one embodiment, the antigen-binding fragment may be an Fab fragment, F(ab′) fragment, F(ab′)2 fragment or Fv fragment. According to one embodiment of the present invention, the antibody may be any one of various types of antibodies capable of binding antigen-specifically, briefly, having antigen-specific binding ability. For example, the antibody may be one in which one light chain and one heavy chain are bonded with each other, or one in which two light chains and two heavy chains are bonded with each other. For example, when two light chains and two heavy chains are bonded with each other, the antibody may be one in which a first unit including a first light chain and a first heavy chain bonded with each other and a second unit including a second light chain and a second heavy chain bonded with each other are combined with each other. Herein, the bond may be a disulfide bond, but it is not limited thereto. According to embodiments of the present invention, the above two units may be the same as or different from each other. For example, the first unit including the first light chain and the first heavy chain and the second unit including the second light chain and the second heavy chain may be the same as or different from each other. As such, an antibody prepared to recognize two different antigens by the first unit and the second unit, respectively, is typically referred to as a ‘bispecific antibody.’ In addition, for example, the antibody may be one in which three or more of the above-described units are combined with one another. The antigen-binding fragment of the present invention may be derived from various types of antibodies as described above, but it is not limited thereto.

According to another embodiment of the present invention, the extracellular domain used herein may be a natural killer receptor (also referred to as a ‘natural cytotoxicity receptor’). According to a specific embodiment, the NK receptor may include NKp46, NKp30, NKp44, NKp80 and NKp65 receptors, but it is not limited thereto.

According to one embodiment of the present invention, the transmembrane domain penetrates through the cell membrane, and may be any one without limitation as long as the domain can penetrate through the cell membrane without interfering with functions of the extracellular domain and the intracellular signaling domain. For example, the transmembrane domain may include the whole or a portion of any one selected from the group consisting of CD8α and CD28.

According to one embodiment of the present invention, the extracellular domain and the transmembrane domain may be linked to each other by a spacer domain. For example, the spacer domain may be a hinge domain. According to a specific embodiment, the spacer domain may include the whole or a portion of any one selected from the group consisting of CD8α and CD28.

According to one embodiment of the present invention, the intracellular signaling domain is a portion located on an inner side of the cell membrane of NK cell, that is, on the cytoplasm thereof, and may include a sequence that can transmit a signal to activate NK cells when an antibody linked to the extracellular domain is combined with a target antigen.

According to one embodiment of the present invention, the chimeric antigen receptor may include one or more intracellular signaling domain(s). When including two or more intracellular signaling domains, the intracellular signaling domains may be linked in series with each other. For example, when including three intracellular signaling domains, a terminal of a first intracellular signaling domain may be linked to a terminal of the transmembrane domain that is not linked with the spacer domain, a terminal of a second intracellular signaling domain may be linked to a terminal of the first intracellular signaling domain that is not linked with the transmembrane domain, and a terminal of a third intracellular signaling domain may be linked to a terminal of the second intracellular signaling domain that is not linked with the first intracellular signaling domain. That is, the first, second and third intracellular signaling domains may be arranged in order from a cell membrane toward an inside of the cell. Further, even when including two, four or more intracellular signaling domains, these domains may be linked to one another in the same manner as described above. According to one embodiment of the present invention, such the respective domains may be directly linked to one another or may be linked by a linker.

According to one embodiment of the present invention, the chimeric antigen receptor may include two intracellular signaling domains. For example, the chimeric antigen receptor may include a first intracellular signaling domain linked to the transmembrane domain and a second intracellular signaling domain linked to a terminal of the first intracellular signaling domain that is not linked with the transmembrane domain. According to a more specific embodiment, the first intracellular signaling domain may include the whole or a portion of any one selected from the group consisting of OX40 (CD134), OX40 ligand (OX40L, CD252), 4-1BB (CD137), CD28, DAP10, CD3-zeta (CD3ζ) and DAP12, and the second intracellular signaling domain may include the whole or a portion of any one selected from the group consisting of OX40 ligand, CD3-zeta and DAP12. In this case, at least one of the first intracellular signaling domain and the second intracellular signaling domain includes the whole or a portion of OX40 ligand. For example, the chimeric antigen receptor may include a first intracellular signaling domain containing the whole or a portion of OX40 ligand and a second intracellular signaling domain containing the whole or a portion of any one selected from CD3-zeta and DAP12. Further, for example, the chimeric antigen receptor may include a first intracellular signaling domain containing the whole or a portion of any one selected from the group consisting of CD3-zeta and DAP12 and a second intracellular signaling domain containing the whole or a portion of OX40 ligand.

According to another embodiment of the present invention, the chimeric antigen receptor may include three intracellular signaling domains. For example, the chimeric antigen receptor may include: a first intracellular signaling domain linked to the transmembrane domain; a second intracellular signaling domain linked to a terminal of the first intracellular signaling domain that is not linked with the transmembrane domain; and a third intracellular signaling domain linked to a terminal of the second intracellular signaling domain that is not linked with the first intracellular signaling domain. According to a more specific embodiment, the first intracellular signaling domain may include the whole or a portion of any one selected from the group consisting of 4-1BB, OX40, OX40 ligand, CD28 and DAP10, the second intracellular signaling domain may include the whole or a portion of any one selected from the group consisting of OX40 ligand, OX40 and 4-1BB, and the third intracellular signaling domain may include the whole or a portion of any one selected from the group consisting of OX40 ligand, CD3-zeta and DAP12. In such a case, at least one of the first intracellular signaling domain, the second intracellular signaling domain and the third intracellular signaling domain may include the whole or a portion of OX40 ligand.

In another aspect, the present invention may provide a chimeric antigen receptor, which includes: a first intracellular signaling domain containing the whole or a portion of any one selected from the group consisting of CD28 and 4-1BB; a second intracellular signaling domain containing the whole or a portion of any one selected from the group consisting of OX40 ligand, OX40 and 4-1BB; and a third intracellular signaling domain containing the whole or a portion of CD3-zeta, wherein the first, second and third intracellular signaling domains are arranged in order from the cell membrane toward the inside of the cell. According to one embodiment of the present invention, the above respective domains may be directly linked to one another or may be linked by a linker.

According to one embodiment of the present invention, the chimeric antigen receptor may further include: a transmembrane domain linked to the first intracellular signaling domain; a spacer domain linked to the transmembrane domain; and an extracellular domain linked to the spacer domain. In addition, the chimeric antigen receptor may further include a signal sequence linked to the extracellular domain. According to one embodiment of the present invention, the above respective domains may be directly linked to one another or may be linked by a linker.

According to one embodiment of the present invention, the extracellular domain is a domain for specifically binding with an antibody or specifically recognizing an antigen, for example, an Fc receptor, an antigen-binding fragment of an antibody such as a single-chain variable fragment (ScFv), NK receptor (natural cytotoxicity receptor), NKG2D, 2B4 or DNAM-1, etc. Thus, in the present disclosure, the term “extracellular domain” is used with the same meanings as the “antigenic recognition site”, “antigen-binding fragment” and/or “antibody binding site.”

The chimeric antigen receptor according to an embodiment of the present invention may include an Fc receptor as the extracellular domain, and therefore, can be used along with a variety of antibodies depending on cell types of cancer to be treated. According to one embodiment, the Fc receptor may include any one selected from the group consisting of CD16, CD32, CD64, CD23 and CD89, and variants thereof. According to a more specific embodiment, the Fc receptor may include CD16 or variants thereof, and most specifically, may include the whole or a portion of CD16 V158 variant (CD16V).

According to another embodiment, the chimeric antigen receptor of the present invention may include, as the extracellular domain, an antigen-binding fragment of an antibody which directly recognizes the antigen without co-administration along with the antibody. According to one embodiment, the antigen-binding fragment may be an Fab fragment, F(ab′) fragment, F(ab′)2 fragment or Fv fragment. According to one embodiment of the present invention, the antibody may be any one of various types of antibodies capable of binding antigen-specifically. For example, the antibody may be one in which one light chain and one heavy chain are bonded with each other, or one in which two light chains and two heavy chains are bonded with each other. For example, when two light chains and two heavy chains are bonded with each other, the antibody may be one in which the first unit including the first light chain and the first heavy chain bonded with each other and the second unit including the second light chain and the second heavy chain bonded with each other are combined with each other. The bond may be a disulfide bond, but it is not limited thereto. According to an embodiment of the present invention, the above two units may be the same as or different from each other. For example, the first unit including the first light chain and the first heavy chain and the second unit including the second light chain and the second heavy chain may be the same as or different from each other. As such, an antibody prepared to recognize two different antigens by the first unit and the second unit, respectively, is commonly referred to as a ‘bispecific antibody’ in the related art. In addition, for example, the antibody may be one in which the above three or more units are combined with one another. The antigen-binding fragment of the present invention may be derived from various types of antibodies as described above, but it is not limited thereto.

According to another embodiment of the present invention, the extracellular domain used herein may be a NK receptor (natural cytotoxicity receptor). According to a specific embodiment, the NK receptor may include NKp46, NKp30, NKp44, NKp80 and NKp65 receptors, but it is not limited thereto.

According to one embodiment, the signal sequence may include the whole or a portion of CD16. According to another embodiment, the extracellular domain may include the whole or a portion of CD16 V158 variant (CD16V). According to another embodiment, the spacer domain may include the whole or a portion of any one selected from the group consisting of CD8α (CD8-alpha) and CD28. According to another embodiment, the transmembrane domain may include the whole or a portion of any one selected from the group consisting of CD8α and CD28.

According to a specific embodiment, the chimeric antigen receptor may include one or more amino acid sequence selected from the group consisting of SEQ ID NOs: 33, 41, 43, 45, 47, 49, 51, 53, 55, 69, 71, 77, 81, 83, 85, 87, 89, 91 and 93 or variants thereof having a sequence identity of 80% or more.

According to another aspect, the present invention provides immune cells (e.g., NK cells) to express the above-described chimeric antigen receptor according to the present invention.

The immune cells of the present invention may exhibit toxicity to tumor cells. It is determined that the chimeric antigen receptor according to the invention exhibits specific toxicity to what types of tumor cells depending on what types of antibodies are combined with the extracellular domains. Therefore, the types of tumor cells, to which the immune cells expressing the chimeric antigen receptor according to the present invention may exhibit specific toxicity, are not particularly limited. According to one embodiment, when the immune cells (e.g., NK cells) of the present invention are used along with rituximab, the cells may exhibit toxicity to malignant lymphoma cells. For example, the malignant lymphoma cells may express CD20. Further, for example, the malignant lymphoma may be B-cell lymphoma.

According to another aspect, the invention further provides a pharmaceutical composition for prevention or treatment of tumor or tumor metastasis, which includes the immune cells (e.g., NK cells) expressing the above-described chimeric antigen receptor according to the present invention, in the number of 2 to 7.5 times the number of tumor cells (e.g., malignant lymphoma cells) in a subject to be treated (‘treatment target’).

According to one embodiment of the present invention, the number of immune cells (e.g., NK cells) included in the pharmaceutical composition of the present invention in a single dose may range from 0.75 to 10 times the number of tumor cells (e.g., malignant lymphoma cells) in the treatment target. For example, the number of the immune cells (e.g., NK cells) in a single dose may range from 2 to 7.5 times the number of tumor cells (e.g., malignant lymphoma cells) in the treatment target.

According to another aspect, the present invention further provides a nucleic acid sequence encoding the above-described chimeric antigen receptor according to the present invention.

According to one embodiment of the present invention, the nucleic acid sequence may include one or more nucleotide sequences selected from the group consisting of SEQ ID NOs: 32, 40, 42, 44, 46, 48, 50, 52, 54, 68, 70, 76, 80, 82, 84, 86, 88, 90 and 92 or variants thereof having a sequence identity of 80% or more.

According to another aspect, the present invention further provides a vector including the above-described nucleic acid sequence according to the present invention.

According to another aspect, the present invention further provides a method of treating tumor which includes administering the above-described immune cells to a subject.

According to another aspect, the present invention further provides a method of preventing tumor metastases which includes administering the above-described immune cells to a subject.

The subject may be a mammal having a tumor, specifically, a human, but it is not limited thereto.

Administration may be performed in such a way that the number of immune cells (e.g., NK cells) expressing the chimeric antigen receptor according to the present invention may be in a range of 2 to 7.5 times the number of tumor cells (e.g., malignant lymphoma cells) in the treatment target.

An administration method is not particularly limited and, for example, may include administration through an oral or parenteral route.

Tumor is not particularly limited but may include, for example, malignant lymphoma, leukemia, breast cancer, lung cancer, etc., and more specifically, B-cell lymphoma.

Hereinafter, the present invention will be described in more detail by way of the following examples. These examples are proposed only for describing the present invention in detail and the scope of the present invention is not limited to the contents illustrated in the examples.

### Example 1: Methods and Reagents

### Cell Lines

The human B-series cell line Ramos, human erythroleukemic cell line K562, human breast cancer cell line MCF-7, H1299 and H1944 of human lung cancer cell lines, as well as NK-92 MI are supplied from American Type Culture Collection (ATCC, Manassas, VA, USA). K562 was maintained in RPMI-1640 containing 10% FBS (Gibco, Grand Island, NY, USA). Ramos was maintained in RPMI-1640 (ATCC) (Manassas, VA) containing 10% FBS (fetal bovine serum; Gibco, Grand Island, NY, USA). MCF-7 was maintained in EMEM (ATCC)+10% FBS (Gibco) medium, while H1299 and H1944 cell lines were maintained in RPMI-1640 (ATCC)+10% FBS (Gibco). In addition, NK-92 MI and transduced NK-92 MI cells were maintained in CellGro® serum-free medium containing 1% human plasma. The human embryonic kidney fibroblast, that is, 293T cell line was supplied from the ATCC. This cell line was maintained in DMEM (Gibco, Grand Island, NY, USA) containing 10% FBS (Gibco, Grand Island, NY, USA).

### Plasmid

The signal sequence and the extracellular domain of the FCRG3A V158 mutant (CD16V); the extracellular domain of NKG2D; the signal sequence of CD8a, the hinge and transmembrane domains of CD8a; the hinge and transmembrane domains of CD28; and the intracellular signaling domains of 4-1BB, OX40, OX40 ligand (OX40L) and CD3ζ, respectively, were synthesized artificially. The above listed materials were assembled in various combinations through splicing by overlapping extension by PCR (‘SOE-PCR’). The PCR products were confirmed by direct sequencing. Each PCR product was cut into Nhe1 and EcoRI, and then inserted into (i.e., ligated to) Nhe1 and EcoRI sites of a 3^{rd} generation self-inactivating lentiviral expression vector such as MSCV-EF1α-GFP vector or EF1a-MCS vector.

Chimeric antigen receptors (CAR) according to embodiments of the present invention have been summarized in Table 1 below. The domains of all CARs according to the embodiments of the present invention have been linked in series (in tandem) to one another and also linked in frame.

TABLE 1

| Type of | | | | | | | | | |
|---|---|---|---|---|---|---|---|---|---|
| CAR | Serial | | Signal | | | | | | |
| (generation) | No. | Abbreviation | sequence | ECD | Hinge | TM | Signal-1 | Signal-2 | Signal-3 |
| — | C1 | NC | CD16 | CD16V | CD8α | CD8α | | | |
| 1st | C2 | CD16V-Z | CD16 | CD16V | CD8α | CD8α | CD3ζ | | |
| 2nd | C3 | CD16V-BBZ | CD16 | CD16V | CD8α | CD8α | 4-1BB | CD3ζ | |
| 2nd | C4 | CD16V-OX40Z | CD16 | CD16V | CD8α | CD8α | OX40 | CD3ζ | |
| 2nd | C5 | CD16V- | CD16 | CD16V | CD8α | CD8α | OX40L | CD3ζ | |
| | | OX40LZ | | | | | | | |
| 2nd | C5-1 | CD16V- | CD16 | CD16V | CD8α | CD8α | CD3ζ | OX40L | |
| | | ZOX40L | | | | | | | |
| 2nd | C6 | CD16V-28Z | CD16 | CD16V | CD8α | CD28 | CD28 | CD3ζ | |
| 2nd | C7 | CD16V-28(H)Z | CD16 | CD16V | CD28 | CD28 | CD28 | CD3ζ | |
| 3rd | C8 | CD16V- | CD16 | CD16V | CD8α | CD8α | 4-1BB | OX40 | CD3ζ |
| | | BBOX40Z | | | | | | | |
| 3rd | C9 | CD16V- | CD16 | CD16V | CD8α | CD8α | OX40 | 4-1BB | CD3ζ |
| | | OX40BBZ | | | | | | | |
| 3rd | C10 | CD16V-28BBZ | CD16 | CD16V | CD8α | CD28 | CD28 | 4-1BB | CD3ζ |
| 3rd | C11 | CD16V- | CD16 | CD16V | CD8α | CD28 | CD28 | OX40 | CD3ζ |
| | | 28OX40Z | | | | | | | |
| 3rd | C12 | CD16V- | CD16 | CD16V | CD8α | CD28 | CD28 | OX40L | CD3ζ |
| | | 28OX40LZ | | | | | | | |
| 3rd | C13 | CD16V- | CD16 | CD16V | CD28 | CD28 | CD28 | 4-1BB | CD3ζ |
| | | 28(H)BBZ | | | | | | | |
| 3rd | C14 | CD16V- | CD16 | CD16V | CD28 | CD28 | CD28 | OX40 | CD3ζ |
| | | 28(H)OX40Z | | | | | | | |
| 3rd | C15 | CD16V- | CD16 | CD16V | CD28 | CD28 | CD28 | OX40L | CD3ζ |
| | | 28(H)OX40LZ | | | | | | | |
| 3rd | C16 | CD16V- | CD16 | CD16V | CD8α | CD8α | 41BB | OX40L | CD3ζ |
| | | BBOX40LZ | | | | | | | |

CD16V-Z CAR (1^{st} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); human CD8α-derived hinge and transmembrane domains (nucleotides 1292-1507, GenBank NM 001768.6); CD3ζ-derived intracellular signaling domain (nucleotides 299-634, GenBank NM 000734.3); and stop codon TGA.

CD16V-BBZ CAR (2^{nd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); human CD8α-derived hinge and transmembrane domains (nucleotides 1292-1507, GenBank NM 001768.6); CD137-derived intracellular signaling domain (nucleotides 901-1026, GenBank NM 001561.5); CD3ζ-derived intracellular signaling domain (nucleotides 299-634, GenBank NM 000734.3); and stop codon TGA.

CD16V-OX40Z CAR (2^{nd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); human CD8α-derived hinge and transmembrane domains (nucleotides 1292-1507, GenBank NM 001768.6); CD134-derived intracellular signaling domain (nucleotides 733-840, GenBank AB590584.1); CD3ζ-derived intracellular signaling domain (nucleotides 299-634, GenBank NM 000734.3); and stop codon TGA.

CD16V-OX40LZ CAR (2^{nd} generation) is provided by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); human CD8α-derived hinge and transmembrane domains (nucleotides 1292-1507, GenBank NM 001768.6); CD252-derived intracellular signaling domain (nucleotides 141-206, GenBank NM 003326.4); CD3ζ-derived intracellular signaling domain (nucleotides 299-634, GenBank NM 000734.3); and stop codon TGA.

CD16V-ZOX40L CAR (2^{nd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); human CD8α-derived hinge and transmembrane domains (nucleotides 1292-1507, GenBank NM 001768.6); CD3ζ-derived intracellular signaling domain (nucleotides 299-634, GenBank NM 000734.3); CD252-derived intracellular signaling domain (nucleotides 141-206, GenBank NM 003326.4); and stop codon TGA. CD16V-28Z CAR (2^{nd} generation) is produced by connecting: the signal sequence domain of CD16 (34-84 nucleotides, GenBank Accession No. X52645); the extracellular domain of CD16V (FCRG3A V158) (nucleotides 85-651, G mutation of nucleotide No. 559 in GenBank Accession No. X52645); human CD8α-derived hinge domain (nucleotides 1292-1435, GenBank NM 001768.6); CD28-derived transmembrane and intracellular signaling domains (nucleotides 679-882, GenBank NM 006139.3); CD32-derived intracellular signaling domain (nucleotides 299-634, GenBank NM 000734.3); and stop codon TGA.

CD16V-28 (H) Z CAR (2^{nd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); CD28-derived hinge, transmembrane and intracellular signaling domains (nucleotides 562-882, GenBank NM 006139.3); CD3ζ-derived intracellular signaling domain (nucleotides 299-634, GenBank NM 000734.3); and stop codon TGA.

CD16V-BBOX40Z CAR (3^{rd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); human CD8α-derived hinge and transmembrane domains (nucleotides 1292-1507, GenBank NM 001768.6); CD137-derived intracellular signaling domain (nucleotides 901-1026, GenBank NM 001561.5); CD134-derived intracellular signaling domain (nucleotides 733-840, GenBank AB590584.1); CD3ζ-derived intracellular signaling domain (nucleotides 299-634, GenBank NM 000734.3); and stop codon TGA.

CD16V-OX40BBZ CAR (3^{rd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); human CD8α-derived hinge and transmembrane domains (nucleotides 1292-1507, GenBank NM 001768.6); CD134-derived intracellular signaling domain (nucleotides 733-840, GenBank AB590584.1); CD137-derived intracellular signaling domain (nucleotides 901-1026, GenBank NM 001561.5); CD3ζ-derived intracellular signaling domain (nucleotides 299-634, GenBank NM 000734.3); and stop codon TGA.

CD16V-28BBZ CAR (3^{rd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); CD8α-derived hinge domain (nucleotides 1292-1435, GenBank NM 001768.6); CD28-derived transmembrane and intracellular signaling domains (nucleotides 679-882, GenBank NM 006139.3); CD137-derived intracellular signaling domain (nucleotides 901-1026, GenBank NM 001561.5); CD3ζ-derived intracellular signaling domain (nucleotides 299-634, GenBank NM 000734.3); and stop codon TGA.

CD16V-28OX40Z CAR (3^{rd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); CD8α-derived hinge domain (nucleotides 1292-1435, GenBank NM 001768.6); CD28-derived transmembrane and intracellular signaling domains (nucleotides 679-882, GenBank NM 006139.3); CD134-derived intracellular signaling domain (nucleotides 733-840, GenBank AB590584.1); CD3č-derived intracellular signaling domain (nucleotides 299-634, GenBank NM 000734.3); and stop codon TGA.

CD16V-28OX40LZ CAR (3^{rd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); CD8α-derived hinge domain (nucleotides 1292-1435, GenBank NM 001768.6); CD28-derived transmembrane and intracellular signaling domains (nucleotides 679-882, GenBank NM 006139.3); CD252-derived intracellular signaling domain (nucleotides 141-206, GenBank NM 003326.4); CD3ζ-derived intracellular signaling domain (nucleotides 299-634, GenBank NM 000734.3); and stop codon TGA.

CD16V-28(H)BBZ CAR (3^{rd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); CD28-derived hinge, transmembrane and intracellular signaling domains (nucleotides 562-882, GenBank NM 006139.3); CD137-derived intracellular signaling domain (nucleotides 901-1026, GenBank NM 001561.5); CD3ζ-derived intracellular signaling domain (nucleotides 299-634, GenBank NM 000734.3); and stop codon TGA.

CD16V-28(H)OX40Z CAR (3^{rd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); CD28-derived hinge, transmembrane and intracellular signaling domains (nucleotides 562-882, GenBank NM 006139.3); CD134-derived intracellular signaling domain (nucleotides 733-840, GenBank AB590584.1); CD3ζ-derived intracellular signaling domain (nucleotides 299-634, GenBank NM 000734.3); and stop codon TGA.

CD16V-28(H)OX40LZ CAR (3^{rd} generation) is produced by connecting: the signal sequence domain of CD16 (nucleotides 34-84, GenBank Accession No. X52645); the extracellular domain of CD16V (nucleotides 85-651, G mutation of nucleotide No. 559 in GenBank Accession No. X52645); CD28-derived hinge, transmembrane and intracellular signaling domains (nucleotides 562-882, GenBank NM 006139.3); CD252-derived intracellular signaling domain (nucleotides 141-206, GenBank NM 003326.4); and CD3ζ-derived intracellular signaling domain (nucleotides 299-634, GenBank NM 000734.3) and stop codon TGA.

CD16V-BBOX40LZ (3^{rd} generation) is produced by connecting: the signal sequence domain of CD16 (nucleotides 34-84, GenBank Accession No. X52645); the extracellular domain of CD16V (FCRG3A V158) (nucleotides 85-651, G mutation of nucleotide No. 559 in GenBank Accession No. X52645); human CD8α-derived hinge and transmembrane domains (nucleotides 1292-1507, GenBank NM 001768.6); CD137-derived intracellular signaling domain (nucleotides 901-1026, GenBank NM 001561.5); CD252-derived intracellular signaling domain (nucleotides 141-206, GenBank NM 003326.4); CD3ζ-derived intracellular signaling domain (nucleotides 299-634, GenBank NM 000734.3); and stop codon TGA.

The sequence listings of the domains used for the chimeric antigen receptors (CARs) and a preparation method thereof according to the embodiments of the present invention are summarized in Table 2 below.

TABLE 2

| Sequence | | |
|---|---|---|
| No. | Name of sequence | Details of sequence |
| 1 | CD16 nucleotide | Signal sequence and extracellular domain of CD16 (34-651 |
| | | nucleotides, GenBank Accession No. X52645) |
| 2 | CD16 amino acid | Amino acid sequence corresponding to SEQ ID NO. 1 |
| 3 | CD16V nucleotide | G mutation of nucleotide No. 559 among signal sequence and |
| | | extracellular domain of CD16V (34-651 nucleotides, |
| | | GenBank Accession No. X52645) |
| 4 | CD16V nucleotide codon | Codon optimized sequence of SEQ ID NO. 3 |
| | optimization | |
| 5 | CD16V amino acid | Amino acid sequence corresponding to SEQ ID NOs. 3 and 4 |
| 6 | CD8α nucleotide | Human CD8α-derived hinge and transmembrane domains |
| | | (1292-1507 nucleotides, GenBank NM 001768.6) |
| 7 | CD8α nucleotide codon | Codon optimized sequence of SEQ ID NO. 6 |
| | optimization | |
| 8 | CD8α amino acid | Amino acid sequence corresponding to SEQ ID NOs. 6 and 7 |
| 9 | CD28 nucleotide | CD28-derived hinge, transmembrane and intracellular |
| | | signaling domains (562-882 nucleotides, GenBank MM |
| | | 006139.3) |
| 10 | CD28 nucleotide codon | Codon optimized sequence of SEQ ID NO. 9 |
| | optimization | |
| 11 | CD28 amino acid | Amino acid sequence corresponding to SEQ ID NOs. 9 and |
| | | 10 |
| 12 | CD3ζ nucleotide | CDζ-derived intracellular signaling domain (299-634 |
| | | nucleotides, GenBank NM 000734.3) |
| 13 | CD3ζ nucleotide codon | Codon optimized sequence of SEQ ID NO. 12 |
| | optimization | |
| 14 | CD3ζ amino acid | Amino acid sequence corresponding to SEQ ID NOs. 12 and |
| | | 13 |
| 15 | OX40L (CD252) nucleotide | OX40L (CD252)-derived intracellular signaling domain (141- |
| | | 206 nucleotides, GenBank NM 003326.4) |
| 16 | OX40L (CD252) nucleotide | Codon optimized sequence of SEQ ID NO. 15 |
| | codon optimization | |
| 17 | OX40L (CD252) amino acid | Amino acid sequence corresponding to SEQ ID NOs. 15 and |
| | | 16 |
| 18 | OX40 (CD134) nucleotide | OX40 (CD134)-derived intracellular signaling domain (733- |
| | | 840 nucleotides, GenBank AB 590584.1) |
| 19 | OX40 (CD134) nucleotide | Codon optimized sequence of SEQ ID NO. 18 |
| | codon optimization | |
| 20 | OX40 (CD134) amino acid | Amino acid sequence corresponding to SEQ ID NOs. 18 and |
| | | 19 |
| 21 | 4-1BB (CD137) nucleotide | 4-1BB (CD137)-derived intracellular signaling domain (901- |
| | | 1026 nucleotides, GenBank NM 001561.5) |
| 22 | 4-1BB (CD137) nucleotide | Codon optimized sequence of SEQ ID NO. 21 |
| | codon optimization | |
| 23 | 4-1BB (CD137) amino acid | Amino acid sequence corresponding to SEQ ID NOs. 21 and |
| | | 22 |
| 24 | CD16V-Z nucleotide | Codon optimized nucleotide sequence of CD16V-Z CAR |
| 25 | CD16V-Z amino acid | Amino acid sequence corresponding to SEQ ID NO. 24 |
| 26 | CD16V-BBZ nucleotide | Codon optimized nucleotide sequence of CD16V-BBZ CAR |
| 27 | CD16V-BBZ amino acid | Amino acid sequence corresponding to SEQ ID NO. 26 |
| 28 | CD16V-OX40Z nucleotide | Codon optimized nucleotide sequence Of CD16V-OX40Z |
| | | CAR |
| 29 | CD16V-OX40Z amino acid | Amino acid sequence corresponding to SEQ ID NO. 28 |
| 30 | CD16V-OX40LZ nucleotide | Codon optimized nucleotide sequence of CD16V-OX30LZ |
| | | CAR |
| 31 | CD16V-OX40LZ amino acid | Amino acid sequence corresponding to SEQ ID NO. 30 |
| 32 | CD16V-28Z nucleotide | Codon optimized nucleotide sequence of CD16V-28Z CAR |
| 33 | CD16V-28Z amino acid | Amino acid sequence corresponding to SEQ ID NO. 32 |
| 34 | CD16V-28(H)Z nucleotide | Codon optimized nucleotide sequence of CD16V-28(H)Z |
| | | CAR |
| 35 | CD16V-28(H)Z amino acid | Amino acid sequence corresponding to SEQ ID NO. 34 |
| 36 | CD16V-BBOX40Z nucleotide | Codon optimized nucleotide sequence of CD16V-BBOX40Z |
| | | CAR |
| 37 | CD16V-BBOX40Z amino acid | Amino acid sequence corresponding to SEQ ID NO. 36 |
| 38 | CD16V-OX40BBZ nucleotide | Codon optimized nucleotide sequence of CD16V-OX40BBZ |
| | | CAR |
| 39 | CD16V-OX40BBZ amino acid | Amino acid sequence corresponding to SEQ ID NO. 38 |
| 40 | CD16V-28BBZ nucleotide | Codon optimized nucleotide sequence of CD16V-28BBZ |
| | | CAR |
| 41 | CD16V-28BBZ amino acid | Amino acid sequence corresponding to SEQ ID NO. 40 |
| 42 | CD16V-28OX40Z nucleotide | Codon optimized nucleotide sequence of CD16V-28OX40Z |
| | | CAR |
| 43 | CD16V-28OX40Z amino acid | Amino acid sequence corresponding to SEQ ID NO. 42 |
| 44 | CD16V-28OX40LZ nucleotide | Codon optimized nucleotide sequence of CD16V-28OX40LZ |
| | | CAR |
| 45 | CD16V-28OX40LZ amino acid | Amino acid sequence corresponding to SEQ ID NO. 44 |
| 46 | CD16V-28(H)BBZ nucleotide | Codon optimized nucleotide sequence of CD16V-28(H)BBZ |
| | | CAR |
| 47 | CD16V-28(H)BBZ amino acid | Amino acid sequence corresponding to SEQ ID NO. 46 |
| 48 | CD16V-28(H)OX40Z | Codon optimized nucleotide sequence of CD16V- |
| | nucleotide | 28(H)OX40Z CAR |
| 49 | CD16V-28(H)OX40Z amino | Amino acid sequence corresponding to SEQ ID NO. 48 |
| | acid | |
| 50 | CD16V-28(H)OX40LZ | Codon optimized nucleotide sequence of CD16V- |
| | nucleotide | 28(H)OX40LZ CAR |
| 51 | CD16V-28(H)OX40LZ amino | Amino acid sequence corresponding to SEQ ID NO. 50 |
| | acid | |
| 52 | CD16V-BBOX40LZ nucleotide | Codon optimized nucleotide sequence of CD16V-BBOX40LZ |
| | | CAR |
| 53 | CD16V-BBOX40LZ amino | Amino acid sequence corresponding to SEQ ID NO. 52 |
| | acid | |
| 54 | CD16V-ZOX40L nucleotide | Codon optimized nucleotide sequence of CD16V-ZOX40L |
| | | CAR |
| 55 | CD16V-ZOX40L amino acid | Amino acid sequence corresponding to SEQ ID NO. 54 |

In addition, the chimeric antigen receptors (CARs) according to the embodiments of the present invention are summarized in Table 3 below. Domains of each of all CARs according to the embodiments of the present invention are linked in series (in tandem) to one other and also linked in the frame.

TABLE 3

| Type of | | | | | | | | | |
|---|---|---|---|---|---|---|---|---|---|
| CAR | Serial | | Signal | | | | | | |
| (generation) | No. | Abbreviation | sequence | ECD | Hinge | TM | Signal-1 | Signal-2 | Signal-3 |
| — | C17 | NC | CD8α | NKG2D | CD8α | CD8α | | | |
| 1st | C18 | NKG2D-Z | CD8α | NKG2D | CD8α | CD8α | CD3ζ | | |
| 2nd | C19 | NKG2D-BBZ | CD8α | NKG2D | CD8α | CD8α | 4-1BB | CD3ζ | |
| 2nd | C20 | NKG2D- | CD8α | NKG2D | CD8α | CD8α | OX40 | CD3ζ | |
| | | OX40Z | | | | | | | |
| 2nd | C21 | NKG2D- | CD8α | NKG2D | CD8α | CD8α | CD3ζ | OX40L | |
| | | OX40L | | | | | | | |
| 2nd | C22 | NKG2D-28Z | CD8α | NKG2D | CD8α | CD28 | CD28 | CD3ζ | |
| 2nd | C23 | NKG2D- | CD8α | NKG2D | CD28 | CD28 | CD28 | CD3ζ | |
| | | 28(H)Z | | | | | | | |
| 3rd | C24 | NKG2D- | CD8α | NKG2D | CD8α | CD8α | 4-1BB | OX40 | CD3ζ |
| | | BBOX40Z | | | | | | | |
| 3rd | C25 | NKG2D- | CD8α | NKG2D | CD8α | CD8α | 4-1BB | OX40L | CD3ζ |
| | | BBOX40LZ | | | | | | | |
| 3rd | C26 | NKG2D- | CD8α | NKG2D | CD8α | CD8α | OX40 | 4-1BB | CD3ζ |
| | | OX40BBZ | | | | | | | |
| 3rd | C27 | NKG2D- | CD8α | NKG2D | CD8α | CD28 | CD28 | 4-1BB | CD3ζ |
| | | 28BBZ | | | | | | | |
| 3rd | C28 | NKG2D- | CD8α | NKG2D | CD8α | CD28 | CD28 | OX40 | CD3ζ |
| | | 28OX40Z | | | | | | | |
| 3rd | C29 | NKG2D- | CD8α | NKG2D | CD8α | CD28 | CD28 | OX40L | CD3ζ |
| | | 28OX40LZ | | | | | | | |
| 3rd | C30 | NKG2D- | CD8α | NKG2D | CD28 | CD28 | CD28 | 4-1BB | CD3ζ |
| | | 28(H)BBZ | | | | | | | |
| 3rd | C31 | NKG2D- | CD8α | NKG2D | CD28 | CD28 | CD28 | OX40 | CD3ζ |
| | | 28(H)OX40Z | | | | | | | |
| 3rd | C32 | NKG2D- | CD8α | NKG2D | CD28 | CD28 | CD28 | OX40L | CD3ζ |
| | | 28(H)OX40LZ | | | | | | | |
| 3rd | C33 | NKG2D-AAA- | CD8α | NKG2D- | CD28 | CD28 | CD28 | OX40L | CD3ζ |
| | | 28(H)OX40LZ | | AAA | | | | | |

NKG2D-Z CAR (1^{st} generation) is produced by connecting: the signal sequence domain of CD8α (nucleotides 890-952, GenBank NM 001768.6); the extracellular domain of NKG2D (nucleotides 788-1192, GenBank ID: AF461811.1); human CD8α-derived hinge and transmembrane domains (nucleotides 1292-1507, GenBank NM 001768.6); CD3ζ-derived intracellular signaling domain (nucleotides 299-634, GenBank NM 000734.3); and stop codon TGA.

NKG2D-BBZ CAR (2^{nd} generation) is produced by connecting: the signal sequence domain of CD8α (nucleotides 890-952, GenBank NM 001768.6); the extracellular domain of NKG2D (nucleotides 788-1192, GenBank ID: AF461811.1); human CD8α-derived hinge and transmembrane domains (nucleotides 1292-1507, GenBank NM 001768.6); CD137-derived intracellular signaling domain (nucleotides 901-1026, GenBank NM 001561.5); CD3ζ-derived intracellular signaling domain (nucleotides 299-634, GenBank NM 000734.3); and stop codon TGA.

NKG2D-OX40Z CAR (2^{nd} generation) is produced by connecting: the signal sequence domain of CD8α (nucleotides 890-952, GenBank NM 001768.6); the extracellular domain of NKG2D (nucleotides 788-1192, GenBank ID: AF461811.1); human CD8α-derived hinge and transmembrane domains (nucleotides 1292-1507, GenBank NM 001768.6); CD134-derived intracellular signaling domain (nucleotides 733-840, GenBank AB590584.1); CD3ζ-derived intracellular signaling domain (nucleotides 299-634, GenBank NM 000734.3); and stop codon TGA.

NKG2D-ZOX40L CAR (2^{nd} generation) is produced by connecting: the signal sequence domain of CD8α (nucleotides 890-952, GenBank NM 001768.6); the extracellular domain of NKG2D (nucleotides 788-1192, GenBank ID: AF461811.1); human CD8α-derived hinge and transmembrane domains (nucleotides 1292-1507, GenBank NM 001768.6); CD3ζ-derived intracellular signaling domain (nucleotides 299-634, GenBank NM 000734.3); CD252-derived intracellular signaling domain (nucleotides 141-206, GenBank NM 003326.4); and CD3ζ stop codon TGA.

NKG2D-28Z CAR (2^{nd} generation) is produced by connecting: the signal sequence domain of CD8α (nucleotides 890-952, GenBank NM 001768.6); the extracellular domain of NKG2D (nucleotides 788-1192, GenBank ID: AF461811.1); human CD8α-derived hinge domain (nucleotides 1292-1435, GenBank NM 001768.6); CD28-derived transmembrane and intracellular signaling domains (nucleotides 679-882, GenBank NM 006139.3); CD3ζ-derived intracellular signaling domain (nucleotides 299-634, GenBank NM 000734.3); and stop codon TGA.

NKG2D-28 (H) Z CAR (2^{nd} generation) is produced by connecting: the signal sequence domain of CD8α (nucleotides 890-952, GenBank NM 001768.6); the extracellular domain of NKG2D (nucleotides 788-1192, GenBank ID: AF461811.1); CD28-derived hinge, transmembrane and intracellular signaling domains (nucleotides 562-882, GenBank NM 006139.3); CD3ζ-derived intracellular signaling domain (nucleotides 299-634, GenBank NM 000734.3); and stop codon TGA.

NKG2D-BBOX40Z CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); human CD8α-derived hinge and transmembrane domains (nucleotides 1292-1507, GenBank NM 001768.6); CD137-derived intracellular signaling domain (nucleotides 901-1026, GenBank NM 001561.5); CD134-derived intracellular signaling domain (nucleotides 733-840, GenBank AB590584.1); CD3 ζ-derived intracellular signaling domain (nucleotides 299-634, GenBank NM 000734.3); and stop codon TGA.

NKG2D-BBOX40LZ CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); human CD8α-derived hinge and transmembrane domains (nucleotides 1292-1507, GenBank NM 001768.6); CD137-derived intracellular signaling domain (nucleotides 901-1026, GenBank NM 001561.5); CD252-derived intracellular signaling domain (nucleotides 141-206, GenBank NM 003326.4); CD3ζ-derived intracellular signaling domain (nucleotides 299-634, GenBank NM 000734.3); and stop codon TGA.

NKG2D-OX40BBZ CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); human CD8α-derived hinge and transmembrane domains (nucleotides 1292-1507, GenBank NM 001768.6); CD134-derived intracellular signaling domain (nucleotides 733-840, GenBank AB590584.1); CD137-derived intracellular signaling domain (nucleotides 901-1026, GenBank NM 001561.5); CD3ζ-derived intracellular signaling domain (nucleotides 299-634, GenBank NM 000734.3); and stop codon TGA.

NKG2D-28BBZ CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); CD8α-derived hinge domain (nucleotides 1292-1435, GenBank NM 001768.6); CD28-derived transmembrane and intracellular signaling domains (nucleotides 679-882, GenBank NM 006139.3); CD137-derived intracellular signaling domain (nucleotides 901-1026, GenBank NM 001561.5); CD3ζ-derived intracellular signaling domain (nucleotides 299-634, GenBank NM 000734.3); and stop codon TGA.

NKG2D-28OX40Z CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); CD8α-derived hinge domain (nucleotides 1292-1435, GenBank NM 001768.6); CD28-derived transmembrane and intracellular signaling domains (nucleotides 679-882, GenBank NM 006139.3); CD134-derived intracellular signaling domain (nucleotides 733-840, GenBank AB590584.1); CD3ζ-derived intracellular signaling domain (nucleotides 299-634, GenBank NM 000734.3); and stop codon TGA.

NKG2D-28OX40LZ CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); CD8α-derived hinge domain (nucleotides 1292-1435, GenBank NM 001768.6); CD28-derived transmembrane and intracellular signaling domains (nucleotides 679-882, GenBank NM 006139.3); CD252-derived intracellular signaling domain (nucleotides 141-206, GenBank NM 003326.4); CD3ζ-derived intracellular signaling domain (nucleotides 299-634, GenBank NM 000734.3); and stop codon TGA.

NKG2D-28(H)BBZ CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); CD28-derived hinge, transmembrane and intracellular signaling domains (nucleotides 562-882, GenBank NM 006139.3); CD137-derived intracellular signaling domain (nucleotides 901-1026, GenBank NM 001561.5); CD3ζ-derived intracellular signaling domain (nucleotides 299-634, GenBank NM 000734.3); and stop codon TGA.

NKG2D-28(H)OX40Z CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); CD28-derived hinge, transmembrane and intracellular signaling domains (nucleotides 562-882, GenBank NM 006139.3); CD134-derived intracellular signaling domain (nucleotides 733-840, GenBank AB590584.1); CD3ζ-derived intracellular signaling domain (nucleotides 299-634, GenBank NM 000734.3); and stop codon TGA.

NKG2D-28(H)OX40LZ CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); CD28-derived hinge, transmembrane and intracellular signaling domains (nucleotides 562-882, GenBank NM 006139.3); CD252-derived intracellular signaling domain (nucleotides 141-206, GenBank NM 003326.4); CD3ζ-derived intracellular signaling domain (nucleotides 299-634, GenBank NM 000734.3); and stop codon TGA.

NKG2D-AAA-28(H)OX40LZ CAR (3^{rd} generation) is produced by connecting: the signal sequence domain of CD8α (nucleotides 890-952, GenBank NM 001768.6); the extracellular domain of NKG2D (nucleotides 788-1192, GenBank ID: AF461811.1); AAA (Triple alanine); CD28-derived hinge, transmembrane and intracellular signaling domains (nucleotides 562-882, GenBank NM 006139.3); CD252-derived intracellular signaling domain (nucleotides 141-206, GenBank NM 003326.4); CD3ζ-derived intracellular signaling domain (nucleotides 299-634, GenBank NM 000734.3); and stop codon TGA.

The sequence listings of the domains used for the chimeric antigen receptors (CARs) and a method for preparation thereof according to the embodiments of the present invention are summarized in Table 4 below.

TABLE 4

| Sequence | | |
|---|---|---|
| No. | Name of sequence | Details of sequence |
| 56 | CD8α nucleotide | Signal sequence domain of CD8α (890-952 nucleotides, |
| | | GenBank NM 001768.6) |
| 57 | CD8α nucleotide codon | Codon optimized sequence of SEQ ID NO. 56 |
| | optimization | |
| 58 | CD8α amino acid | Amino acid sequence corresponding to SEQ ID NOs. 56 and |
| | | 57 |
| 59 | NKG2D nucleotide | Extracellular domain of NKG2D (788-1192 nucleotides, |
| | | GenBank ID: AF 461811.1) |
| 60 | NKG2D nucleotide codon | Codon optimized sequence of SEQ ID NO. 59 |
| | optimization | |
| 61 | NKG2D amino acid | Amino acid sequence corresponding to SEQ ID NOs. 59 and |
| | | 60 |
| 62 | NKG2D-Z nucleotide | Codon optimized nucleotide sequence of NKG2D-Z CAR |
| 63 | NKG2D-Z amino acid | Amino acid sequence corresponding to SEQ ID NO. 62 |
| 64 | NKG2D-BBZ nucleotide | Codon optimized nucleotide sequence of NKG2D-BBZ CAR |
| 65 | NKG2D-BBZ amino acid | Amino acid sequence corresponding to SEQ ID NO. 64 |
| 66 | NKG2D-OX40Z nucleotide | Codon optimized nucleotide sequence of NKG2D-OX40Z |
| | | CAR |
| 67 | NKG2D-OX40Z amino acid | Amino acid sequence corresponding to SEQ ID NO. 66 |
| 68 | NKG2D-ZOX40L nucleotide | Codon optimized nucleotide sequence of NKG2D-ZOX40L |
| | | CAR |
| 69 | NKG2D-ZOX40L amino acid | Amino acid sequence corresponding to SEQ ID NO. 68 |
| 70 | NKG2D-28Z nucleotide | Codon optimized nucleotide sequence of NKG2D-28Z CAR |
| 71 | NKG2D-28Z amino acid | Amino acid sequence corresponding to SEQ ID NO. 70 |
| 72 | NKG2D-28(H)Z nucleotide | Codon optimized nucleotide sequence of NKG2D-28(H)Z |
| | | CAR |
| 73 | NKG2D-28(H)Z amino acid | Amino acid sequence corresponding to SEQ ID NO. 72 |
| 74 | NKG2D-BBOX40Z nucleotide | Codon optimized nucleotide sequence of NKG2D-BBOX40Z |
| | | CAR |
| 75 | NKG2D-BBOX40Z amino acid | Amino acid sequence corresponding to SEQ ID NO. 74 |
| 76 | NKG2D-BBOX40LZ | Codon optimized nucleotide sequence of NKG2D- |
| | nucleotide | BBOX40LZ CAR |
| 77 | NKG2D-BBOX40LZ amino | Amino acid sequence corresponding to SEQ ID NO. 76 |
| | acid | |
| 78 | NKG2D-OX40BBZ nucleotide | Codon optimized nucleotide sequence of NKG2D-OX40BBZ |
| | | CAR |
| 79 | NKG2D-OX40BBZ amino acid | Amino acid sequence corresponding to SEQ ID NO. 78 |
| 80 | NKG2D-28BBZ nucleotide | Codon optimized nucleotide sequence of NKG2D-28BBZ |
| | | CAR |
| 81 | NKG2D-28BBZ amino acid | Amino acid sequence corresponding to SEQ ID NO. 80 |
| 82 | NKG2D-280X40Z nucleotide | Codon optimized nucleotide sequence of NKG2D-28OX40Z |
| | | CAR |
| 83 | NKG2D-280X40Z amino acid | Amino acid sequence corresponding to SEQ ID NO. 82 |
| 84 | NKG2D-280X40LZ nucleotide | Codon optimized nucleotide sequence of NKG2D-28OX40LZ |
| | | CAR |
| 85 | NKG2D-280X40LZ amino | Amino acid sequence corresponding to SEQ ID NO. 84 |
| | acid | |
| 86 | NKG2D-28(H)BBZ nucleotide | Codon optimized nucleotide sequence of NKG2D-28(H)BBZ |
| | | CAR |
| 87 | NKG2D-28(H)BBZ amino acid | Amino acid sequence corresponding to SEQ ID NO. 86 |
| 88 | NKG2D-28(H)OX40Z | Codon optimized nucleotide sequence of NKG2D- |
| | nucleotide | 28(H)OX40Z CAR |
| 89 | NKG2D-28(H)OX40Z amino | Amino acid sequence corresponding to SEQ ID NO. 88 |
| | acid | |
| 90 | NKG2D-28(H)OX40LZ | Codon optimized nucleotide sequence of NKG2D- |
| | nucleotide | 28(H)OX40LZ CAR |
| 91 | NKG2D-28(H)OX40LZ amino | Amino acid sequence corresponding to SEQ ID NO. 90 |
| | acid | |
| 92 | NKG2D-AAA-28(H)OX40LZ | Codon optimized nucleotide sequence of NKG2D-AAA- |
| | Nucleotide | 28(H)OX40LZ CAR |
| 93 | NKG2D-AAA-28(H)OX40LZ | Amino acid sequence corresponding to SEQ ID NO. 92 |
| | amino acid | |

### Virus Production and Gene Transfer

In order to prepare VSVG-pseudotyped lentivirus, 293T cells cultured in a DMEM medium were co-transfected with various types of vectors such as PCDH1-MSCV-CD16-construct-EF1-copGFP vector, EF1a-NKG2D-construct vector, PCDH1-MSCV-EF1-copGFP control vector, or EF1a-GFP control vector (for production of Mock infection virus using empty vector) together with HIV-based pPACKH1 lentivirus Package Kit (System Biosciences). For this purpose, Lipofectamine 2000 reagent (Invitrogen, Carlsbad, CA) was used. Various types of CD16V constructs are as follows; CD16V-Z CAR, CD16V-BBZ CAR, CD16V-OX40Z CAR, CD16V-OX40LZ CAR, CD16V-ZOX40L, CD16V-28Z CAR, CD16V-28 (H) Z CAR, CD16V-BBOX40Z CAR, CD16V-BBOX40LZ CAR, CD16V-OX40BBZ CAR, CD16V-28BBZ CAR, CD16V-28OX40Z CAR, CD16V-28OX40LZ CAR, CD16V-28(H)BBZ CAR, CD16V-28(H)OX40Z CAR, CD16V-28(H)OX40LZ CAR. Further, various types of NKG2D constructs are as follows; NKG2D-Z CAR, NKG2D-BBZ CAR, NKG2D-OX40Z CAR, NKG2D-ZOX40L CAR, NKG2D-28Z CAR, NKG2D-28 (H) Z CAR, NKG2D-BBOX40Z CAR, NKG2D-BBOX40LZ CAR, NKG2D-OX40BBZ CAR, NKG2D-28BBZ CAR, NKG2D-28OX40Z CAR, NKG2D-28OX40LZ CAR, NKG2D-28(H)BBZ CAR, NKG2D-28(H)OX40Z CAR, NKG2D-28(H)OX40LZ CAR, NKG2D-AAA-28(H)OX40LZ. Each lentivirus was prepared by transfection of 80% dense HEK293T cells in a flask with: various types of CD16V construct expression vectors, various types of NKG2D construct expression vectors or a control plasmid together with pPACKH1 lentivirus packaging plasmids. After 6 hours, the medium was replaced by a DMEM medium containing 10% FBS. The conditioned medium containing lentivirus was collected after 48 hours of transfection, followed by filtering with a 0.45 μm filter unit (Milliopore, Billerica, MA, USA) in order to remove cell debris. A viral supernatant containing the virus was concentrated about 50 times by centrifugation at 3000 rpm and 4° C. for 20 minutes using Amicon Filter (Millipore). The concentrated virus was stored at −80° C.

For the lentiviral infection, NK92MI cells in an exponential growth phase was adjusted to a concentration of 1×106 cells/ml using Cellgro (Cellgenix) including 1% human plasma, and then a lentiviral supernatant in 50 to 100 MOI was added in the presence of 8 μg/ml polybrene, followed by centrifugation at 1800 g for 90 minutes. After centrifugation, the cells were left in a humidified incubator at 37° C. and 5% CO2 conditions for 48 hours. Thereafter, the cells were washed twice with RPMI-1640, and then left in RPMI-1640 including 10% FBS for future use. Control cells were transduced with a vector only.

### Detection of Expression of Receptor Including CD16V or NKG2D

CD16V CAR-transduced NK92MI cells, NKG2D CAR-transduced NK92MI cells, the control vector-transduced NK92MI (NK92MI-Mock) or NK-92 MI parent cells were washed twice with FACS buffer, and the washed cells were stained using 7-AAD (Beckman coulter), anti-CD3, anti-CD56 and anti-CD16 (BD Biosciences) mAbs. An expression ratio and a mean fluorescence intensity (MFI) of the stained cells were measured using a BD LSRFortessa.

The transduction efficiency using the NKG2D construct was determined by flow cytometric analysis of cells expressing NKG2D among CD3-CD56+ cells. First, NK92MI cells were gated in regard to singlet, and then gated in regard to 7AAD- and CD3-CD56+. The transformation efficiency using the CD16 constructs was determined by flow cytometric analysis of cells expressing GFP and CD16 among CD3-CD56+ cells.

### Calcein Releasing Cytotoxicity Assay

Target cells were labeled at 37° C. for 1 hour with 30 μM calcein-acetoxymethyl ester (Calcein-AM; Molecular probes). After washing, the labeled target cells were dispensed to 1×10⁴ cells per well in 96-well plates. NK92MI cells were harvested, washed, and then were added at different E/T (effector-to-target) ratios under conditions with or without rituximab at various concentrations. As a control group independent of the rituximab, an anti-human antibody (Sigma aldrich) was used. After 2 hours, the plates were centrifuged at 2000 rpm for 3 minutes, and a supernatant of 100 μL was collected and subjected to measurement of calcein release using a fluorescence microplate reader (Victor3, PerkinElmer) at an excitation wavelength of 485 nm and an emission wavelength of 535 nm. Specific calcein release amount was calculated by the following equation: percent specific lysis=(test release−spontaneous release)×100/(maximal release−spontaneous release). For maximal lysis, a 1% Triton X-100 was used.

### Example 2: Assessment of Cytotoxicity of NK92MI Cells Expressing OX40 Ligand (CD252)-Containing Chimeric Antigen Receptor (CAR) Against CD20-Positive Lymphoma Cells

Transduction and Expression of Chimeric Antigen Receptor Containing CD16V Associated with the Co-Stimulating Motif

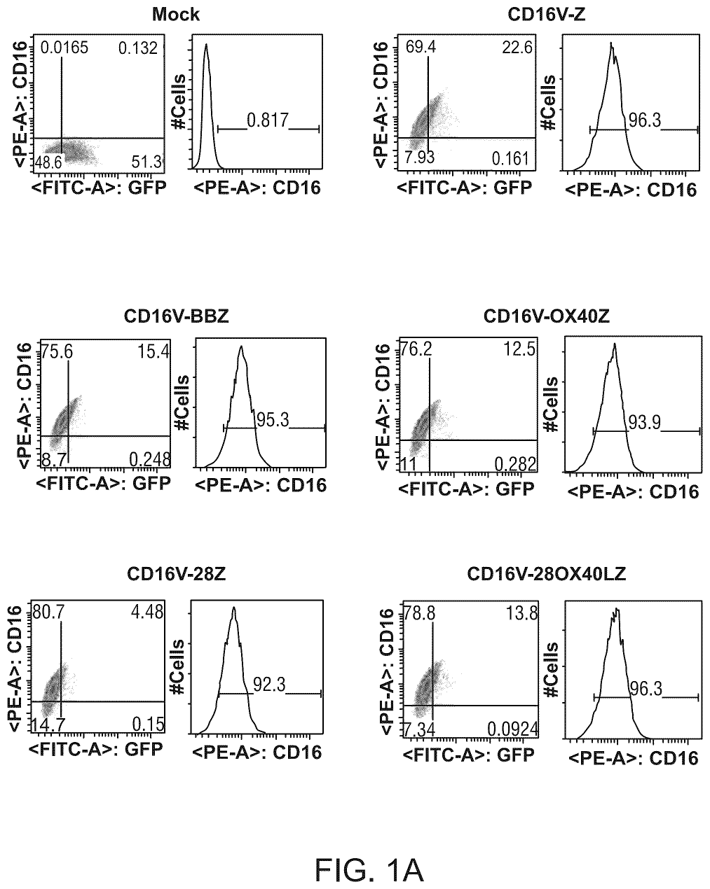

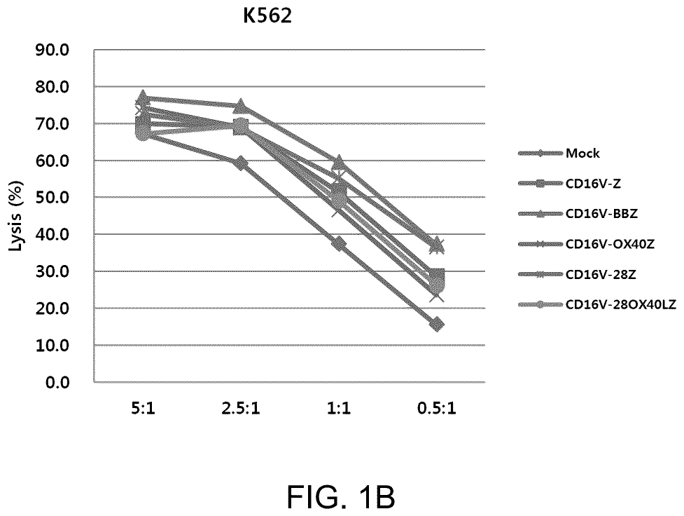

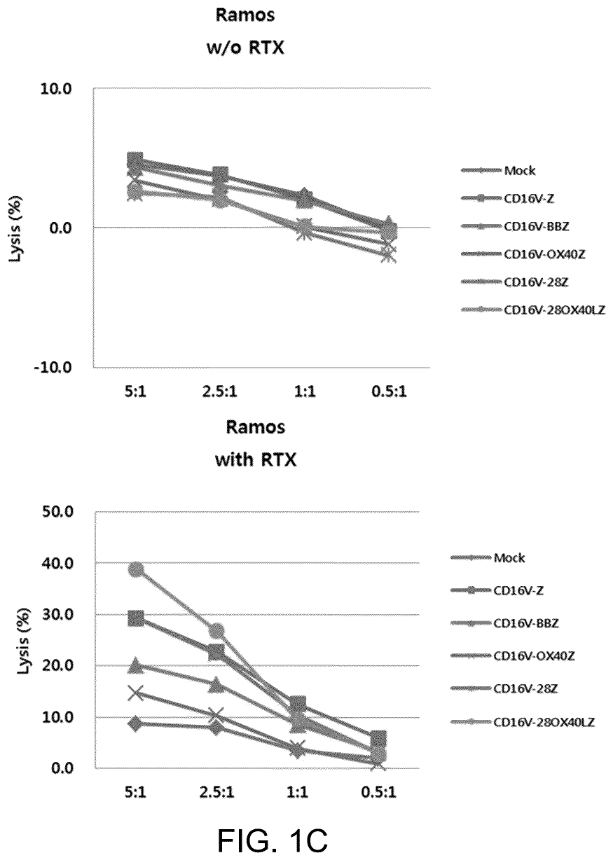

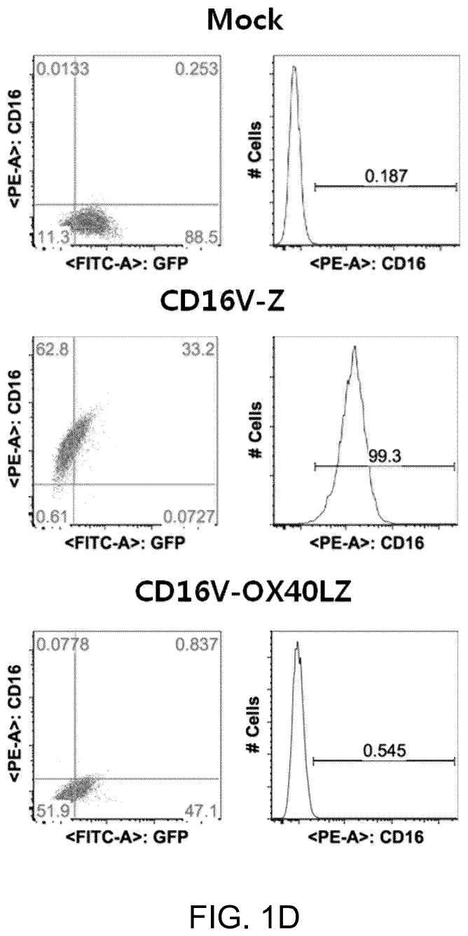

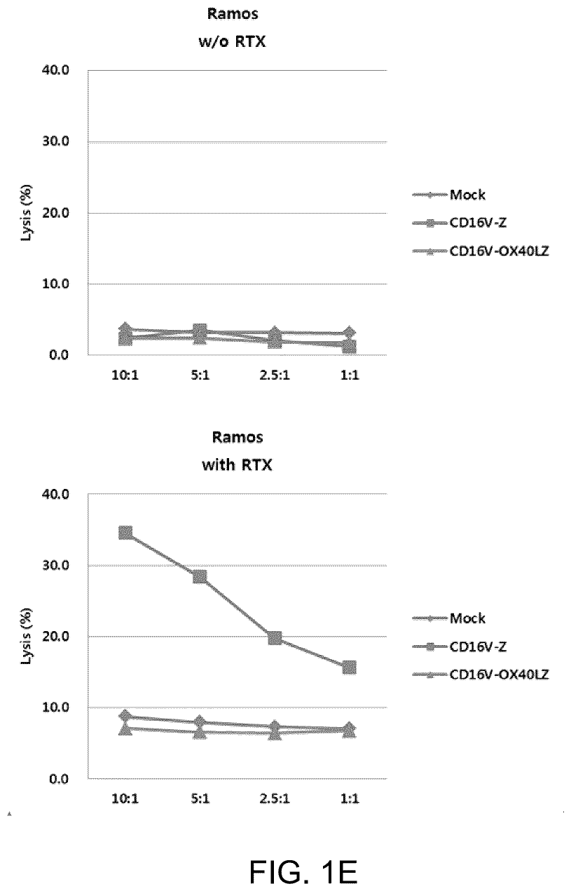

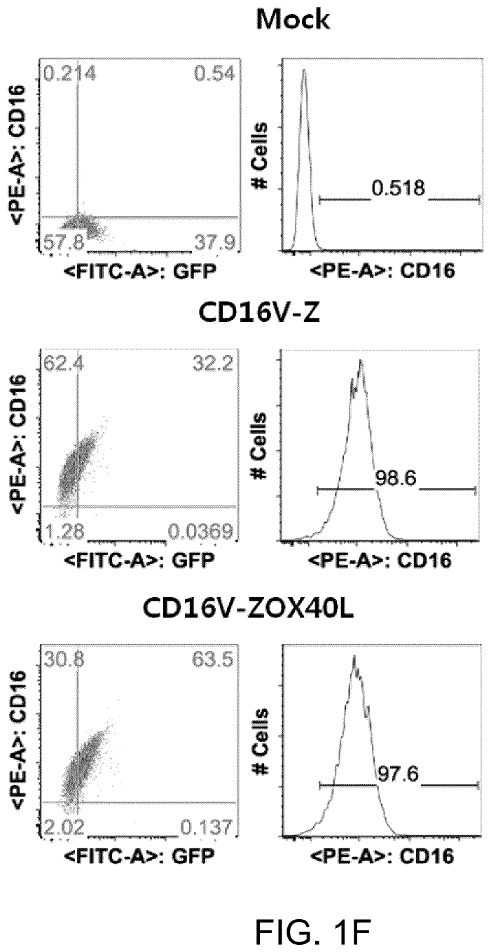

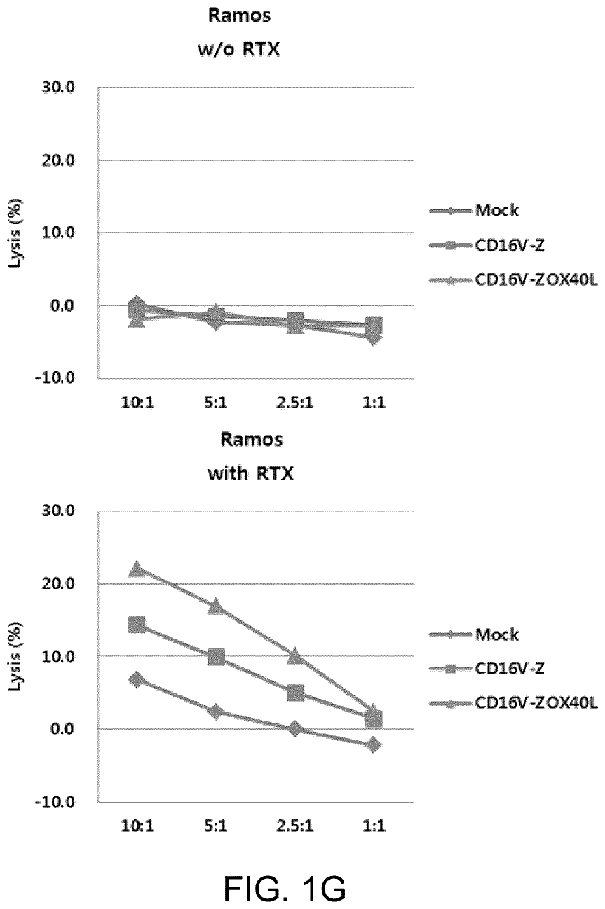

V158 variant (polymorphism) of FCRG3A (CD16) is a high affinity immunoglobulin Fc receptor and is considered to exhibit good effects in antibody treatment. The present inventors have prepared the V158 variant of FCRG3A (CD16) and combined the prepared variant with: the hinge and transmembrane domains of CD8α; a T cell stimulatory molecule, that is, CD3ζ; and intracellular domains of different costimulatory molecules such as CD28, 4-1BB, OX-40 and OX-40 ligand in various combinations thereof (Table 1). The prepared CD16V-containing chimeric antigen receptors (e.g., CD16V-Z CAR (1^{st} generation), CD16V-28Z CAR (2^{nd} generation), CD16V-BBZ CAR (2^{nd} generation), CD16V-OX40Z CAR (2^{nd} generation) or CD16V-28OX40LZ CAR (3^{rd} generation)) were expressed in NK92MI cells by a lentiviral vector including a MSCV promoter. We confirmed surface expression of each CAR on NK92MI cells by detection of the human CD16, using a monoclonal mouse anti-human antibody. By repetitive experiments involving flow cytometric analysis, it was demonstrated that CARs were transduced with efficiency more than 90% in NK92MI cells (FIG. 1A). An amount of lentivirus used herein is equal to the multiple of infection (MOI) of 50 or more.

Increase of Tumor Killing Effect of NK92MI Cells Expressing CD16V Receptor Combined with CD252 (OX40 Ligand) Against CD20-Positive Lymphoma

In order to determine whether genetic modification according to the invention induces an increase in cancer cell death, cytotoxicity of NK92MI cells transduced with an empty vector and NK92MI cells expressing a CD16-containing receptor on CD20-positive lymphoma cells (Ramos cells) was evaluated by calcein-AM release assay.

Before test for lymphoma cells, K562, a human erythroleukemic cell line, was used as a standard control to assess intrinsic cell killing ability (i.e., cytotoxicity) of the transduced NK92MI cells. By transduction using lentivirus having multiple of infection (MOI) of 50 or more, each of CARs according to the present invention (CD16V-Z CAR (1^{st} generation), CD16V-28Z CAR (2^{nd} generation), CD16V-BBZ CAR (2^{nd} generation), CD16V-OX40Z CAR (2^{nd} generation) or CD16V-28OX40LZ CAR (3^{rd} generation)) was expressed at a high level in 90% or more NK92MI cells (FIG. 1A).

It was demonstrated that cytotoxicity of NK92MI cells expressing CARs according to the invention on K562 was similar to cytotoxicity of a control group (Mock) transduced with an empty vector. This means that the intrinsic cytotoxicity of NK92MI cells on a target was not increased by side effects due to the genetic modification itself (FIG. 1B). In FIG. 1B, a ratio of the number of NK92MI cells as effector cells to the number of K562 cells as target cells is indicated by 5:1, 2.5:1, 1:1 and 0.5:1.

In order to assess how diverse co-stimulatory molecules affect in vitro on antigen-specific antitumor activity of NK92MI cells expressing CD16V-containing receptor, transduced NK92MI cells and cancer cells (B-cell lymphoma cell line, that is, Ramos) were subjected to co-incubation, followed by assessment of lysis of the cancer cells according to calcein-AM release assay (FIG. 1C). In FIG. 1C, ratios of the number of NK92MI cells (effector cells) to the number of Ramos cells (target cells) are indicated by 5:1, 2.5:1, 1:1 and 0.5:1. It has been shown that rituximab does not increase NK-92 MI mediated cytotoxicity significantly because NK92MI does not express CD16.

As shown in FIG. 1C, we found that expression of various CARs according to the present invention without rituximab did not increase significantly NK-92 MI mediated cytotoxicity over that cytotoxicity induced by the control (Mock), NK-92 MI cells transduced with the vector containing only GFP.

CD16V-Z CAR (first generation), which was previously shown to enhance cytotoxicity in various CAR applications, served as positive control. In order to increase cytotoxicity of the CD16V-containing receptor, the present inventors have introduced CD28, CD134 (OX40), CD137 (4-1BB) or CD252 (OX40 ligand) into CD16V-Z-CAR. In 2 hour in vitro cytotoxicity assays, NK-92 MI cells expressing CD16V-Z CAR (1^{st} generation), CD16V-28Z CAR (2^{nd} generation), CD16V-BBZ CAR (2^{nd} generation), CD16V-OX40Z CAR (2^{nd} generation) or CD16V-28OX40LZ CAR (3^{rd} generation) exhibited high cytotoxicity in the presence of rituximab at effector cells to target cells ratio of 5:1 and 2.5:1. Among them, in particular, 3^{rd} generation CAR including OX40 ligand, that is, CD16V-28OX40LZ CAR exhibited the highest cytotoxicity, compared to the other CARs (FIG. 1C).

In order to investigate whether 2^{nd} generation CAR containing OX40 ligand can enhance anticancer activity of NK92MI cells, CD16V-OX40LZ CAR was prepared and subjected to evaluation of cytotoxicity. When NK92MI cells were transduced with CD16-OX40LZ using a lentiviral vector, CD16V-OX40LZ CAR was not expressed (FIG. 1D). Even in the presence of rituximab, cytotoxicity on Ramos cells mediated by CD16V-OX40LZ CAR was not exhibited (FIG. 1E). Accordingly, the present inventors noticed a fact that OX40 ligand is type II protein and CD3ζ linked to the OX40 ligand is type I protein, and has prepared CD16V-ZOX40L CAR having CD3ζ at N-terminal and OX40 ligand at C-terminal by changing the order of the above two domains. When NK92MI cells were transduced with CD16V-ZOX40L using a lentiviral vector having an MSCV promoter, NK92MI cells effectively expressed CD16V-OX40LZ CAR compared with CD16V-OX40LZ (FIG. 1F). Further, as a result of assessing anticancer activity of 2^{nd} generation CAR having CD16V-ZOX40L introduced therein, high cytotoxicity on Ramos cells was demonstrated at ratios of effector cells to target cells of 10:1, 5:1 and 2.5:1 in the presence of rituximab (FIG. 1G). It was determined that, in addition to CD16V-28OX40LZ CAR (3^{rd} generation) containing OX40 ligand, CD16V-ZOX40L CAR (2^{nd} generation) also has anticancer activity superior to CD16V-Z CAR used as a positive control.

### Assessment of Efficacy of CD16V-BBOX40LZ CAR

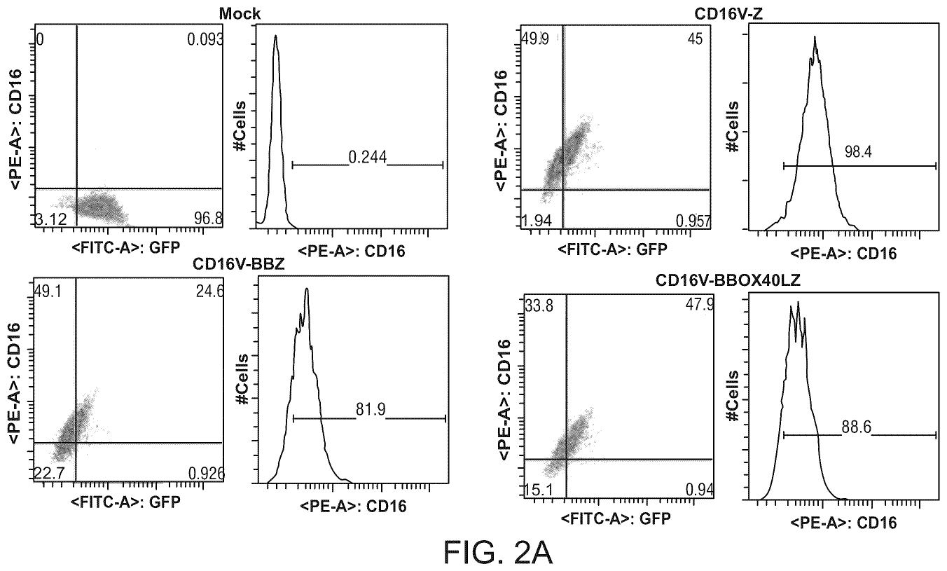

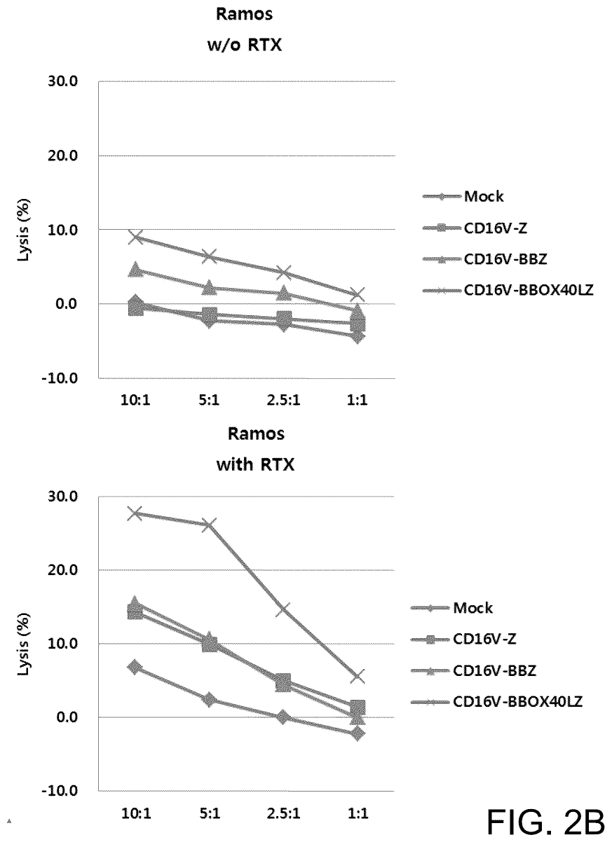

By introducing OX40 ligand into CD137 (41BB) signaling domain-based CAR, NK92MI cells were subjected to assessment of expression and cytotoxicity. Both of CD16V-BBZ CAR (2^{nd} generation) and CD16V-BBOX40LZ CAR (3^{rd} generation) were expressed at high levels in NK92MI cells (FIG. 2A). When rituximab is not present in an in vitro test for cytotoxicity, all of CD16V-Z, CD16V-BBZ and CD16V-BBOX40LZ exhibited low cytotoxicity to Ramos cells. On the other hand, in the presence of rituximab, CD16V-BBOX40LZ in combination with OX40 ligand exhibited highest cytotoxicity at even any ratio of effector cells to target cells (FIG. 2B).

### Comparison of Efficacies Between CD16V-28OX40LZ CAR and Other CARs

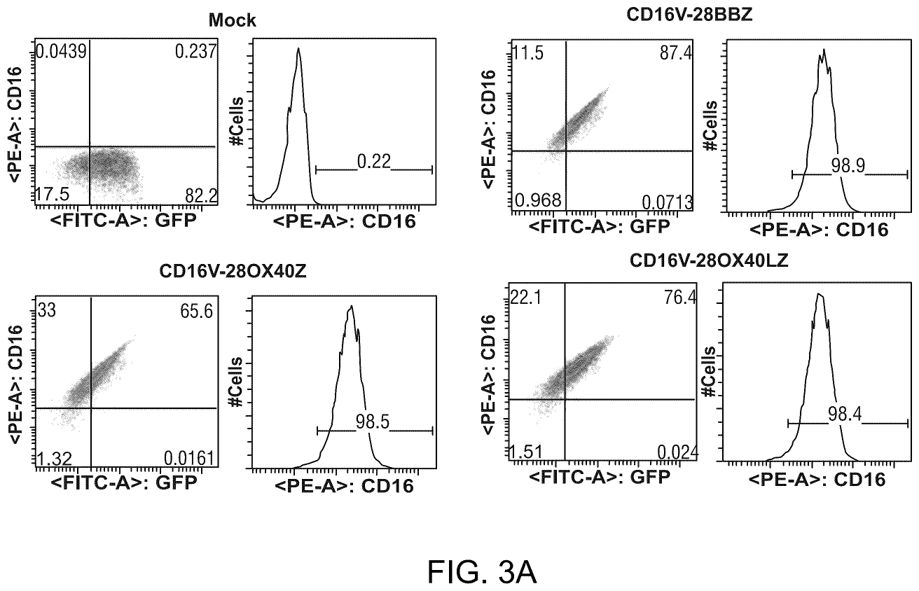

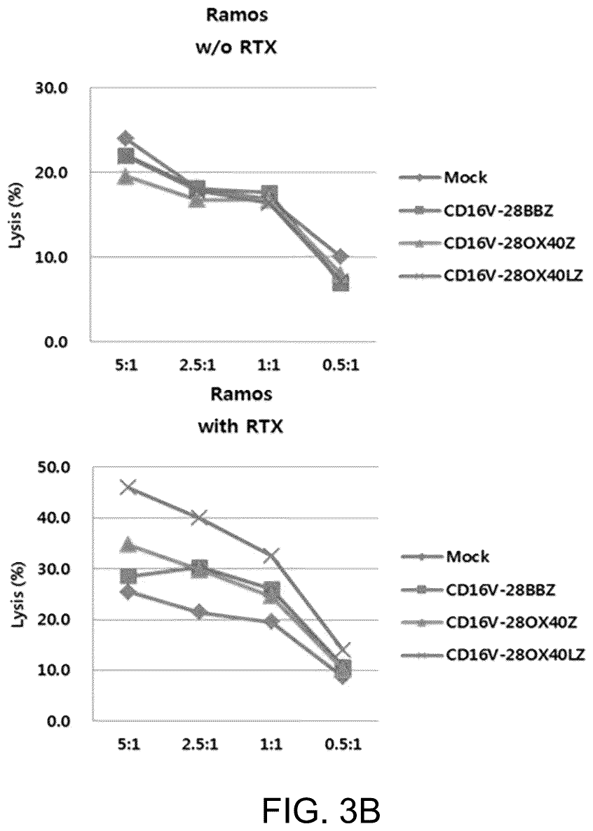

Next, the present inventors have compared the cytotoxicity of NK92MI cells expressing CD16V-28OX40LZ CAR and the cytotoxicity of NK92MI cells expressing 3^{rd} generation CAR that has other intracellular signaling domains. The 3^{rd} generation CARs used in the present experiment (CD16V-28OX40LZ CAR, CD16V-28OX40Z CAR and CD16V-28BBZ CAR) were all proven to be expressed at high levels in NK92MI cells (FIG. 3A).

After comparing NK cell activating efficacies of various 3^{rd} generation CARs (that is, 3^{rd} generation chimeric antigen receptors) according to the present invention, results thereof are summarized in FIG. 3B. As shown in FIG. 3B, all of these CARs show low cytotoxicity to Ramos cells when rituximab is not present, and an extent of the cytotoxicity is all similar. On the other hand, in the presence of rituximab, all of CD16V-28OX40LZ CAR, CD16V-28OX40Z CAR and CAR CD16V-28BBZ showed high cytotoxicity to Ramos cells. Among them, in particular, CD16V-28OX40LZ CAR containing OX40 ligand exhibited the most excellent cytotoxicity (FIG. 3B).

### Assessment of Efficacy of 3^{rd} Generation CAR Including OX40 Ligand Having CD28 at Hinge

In order for the CAR expressing NK cells to recognize a cancer cell antigen in an optimized condition, not only cancer cell antigen-recognizing receptor itself, but also hinge sequence and composition between an antigen-specific receptor and a cell membrane are also important. The hinge sequence and composition may need to be designed differently depending on target molecules.

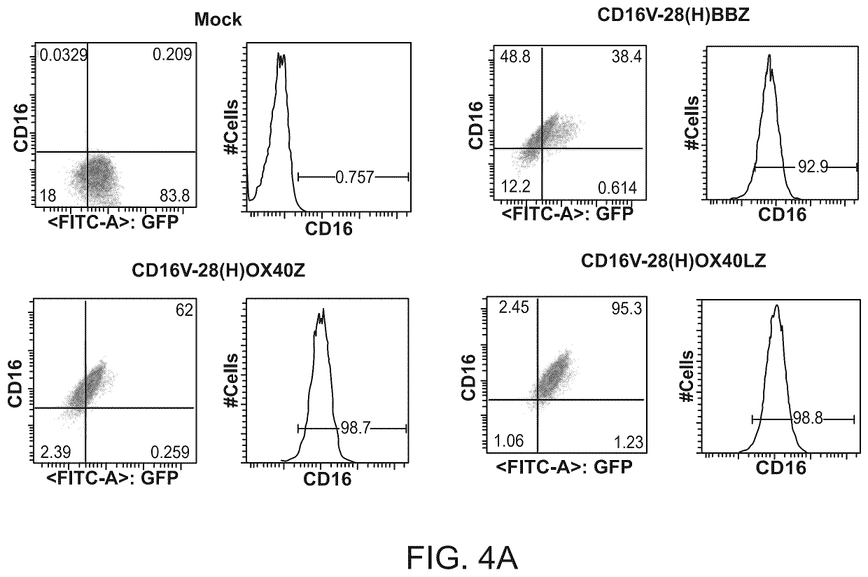

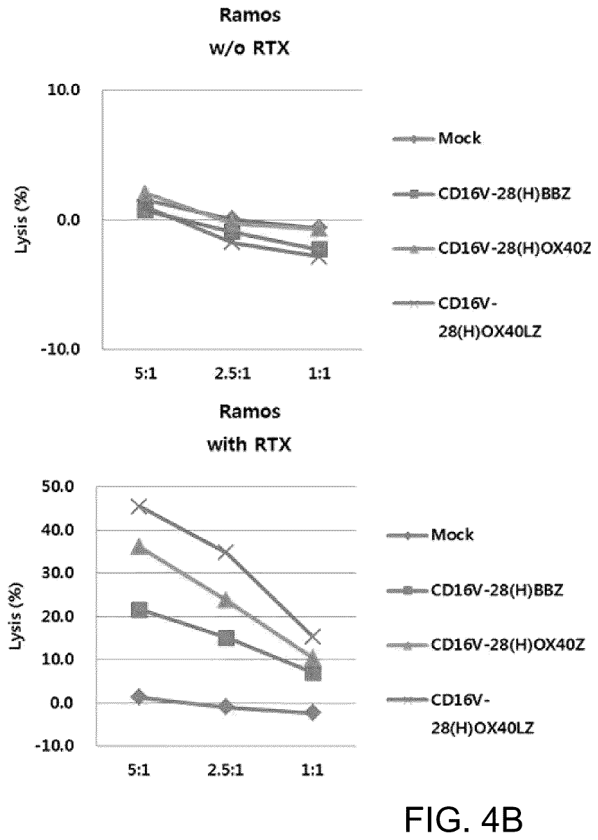

The CARs used in the experiments described above use the fragment of CD8α as spacer domains (‘hinge’). As compared to this, variation of cytotoxicity of CARs when the fragment of CD28 is used at the hinge, was assessed and results thereof are summarized in FIG. 4. The present inventors have produced a lentiviral vector including an extracellular domain (ectodomain) of CD16V and the hinge of CD28. In 3^{rd} generation CARs containing CD28 fragment as spacer, CD28-derived hinge, transmembrane and intracellular signaling domains were linked to a signaling module of CD134 (OX40), CD137 (4-1BB) or OX40 ligand (CD252). Such produced 3^{rd} generation CARs were expressed on NK92MI cells using the lentiviral vector. It was demonstrated that the transduced NK92MI cells could express the above 3^{rd} generation CARs, that is, CD16V-28(H)BBZ CAR, CD16V-28(H)OX40Z CAR or CD16V-28(H)OX40LZ CAR) at high levels, respectively (FIG. 4A).

As shown in FIG. 4B, when rituximab is not present, all of CD16V-28(H)BBZ CAR, CD16V-28(H)OX40Z CAR and CD16V-28(H)OX40LZ CAR do not exhibit cytotoxicity. On the other hand, in the presence of rituximab, all of CD16V-28(H)BBZ CAR, CD16V-28(H)OX40Z CAR and CD16V-28(H)OX40LZ CAR exhibit high cytotoxicity. Among them, in particular, CD16V-28(H)OX40LZ CAR including OX40 ligand has the highest cytotoxicity.

The experimental results described above demonstrate that, when a novel CAR particularly including OX40 ligand as an intracellular signaling domain discovered by the present inventors is expressed in natural killer cells, excellent antitumor effects may be attained.

### Example 3: Assessment of Cytotoxicity of NK92MI Cells Expressing OX40 Ligand (CD252)-Containing NKG2D Chimeric Antigen Receptor (NKG2D-CAR) on Human Breast Cancer Cells and Lung Cancer Cells

Transduction and Expression of a Chimeric Antigen Receptor Including NKG2D Associated with Co-Stimulatory Motif

The present inventors synthesized a human NKG2D gene and combined the same with diverse combinations of: hinge and transmembrane domains of CD8α; and intracellular domains of T-cell stimulatory molecules CD3ζ and cofactors including CD28, 4-1BB, OX-40 and OX-40 ligand to significantly enhance activity of T or NK cells (Table 3). Such NKG2D CAR constructs were expressed in NK92MI cells using a lentiviral vector. The present inventors have identified surface expression of each NKG2D CAR in NK92MI cells by means of detection of human NKG2D using a monoclonal mouse anti-human antibody. By repetitive experiments involving flow cytometric analysis, it was demonstrated that CARs were transduced with 70% or more efficiency in NK92MI cells.

Increase of Tumor Killing Effect of NK92MI Cells Expressing NKG2D Receptor, which Contains Various Co-Stimulatory Signaling Domains, Against MCF7 Breast Cancer Cell Line

In order to investigate variation in killing activity due to genetic modification, cytotoxicity of NK-92 MI cells, which express NKG2D CAR, on MCF7 breast cancer cells was compared by calcein-AM release assay.

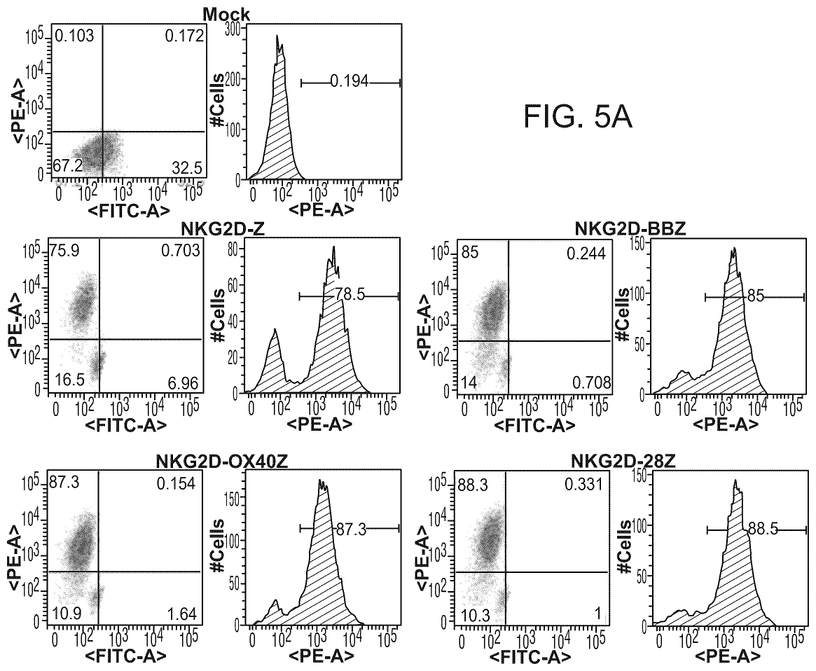

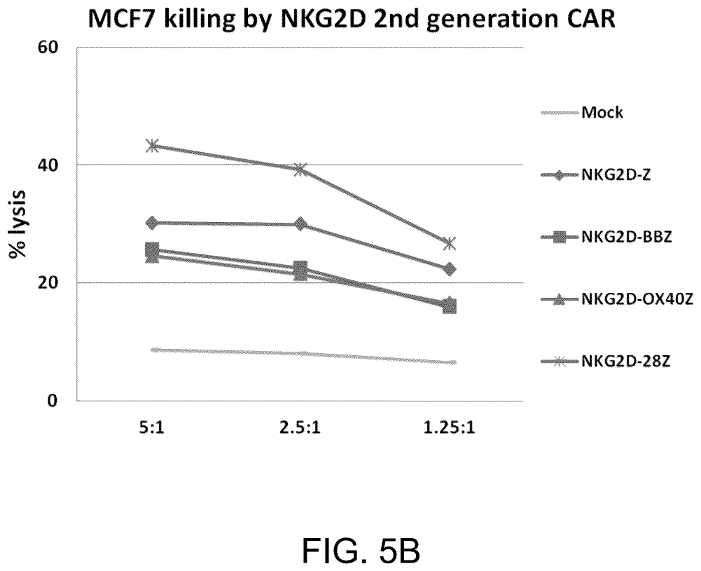

In order to assess in vitro effects of various co-stimulatory signaling domains on antigen-specific antitumor functions of NK-92 MI cells expressing NKG2D CAR, transduced NK-92 MI cells and cancer cells were co-cultured, followed by measuring lysis of tumor cells through calcein-AM release. According to previous studies, it was known that adding the co-stimulatory signaling domains to the chimeric receptor exhibits increased cytotoxicity of T and NK lymphocytes. In order to increase the cytotoxicity of NKG2D CAR, the present inventors have introduced signaling domains of the most well-known three cofactors, that is, CD28, CD134 (OX-40) and CD137 (4-1BB) into the NKG2D CAR. The NK92MI cells containing the introduced NKG2D-Z CAR (1^{st} generation), NKG2D-28Z CAR, NKG2D-BBZ CAR, NKG2D-OX40Z CAR (2^{nd} generation) showed efficient expression of NKG2D (FIG. 5A). In vitro cytotoxicity analysis for two hours, it was demonstrated that NK-92 MI cells expressing NKG2D-Z CAR (1^{st} generation), NKG2D-28Z CAR, NKG2D-BBZ CAR and NKG2D-OX40Z CAR (2^{nd} generation) exhibited cytotoxicity to MCF7 breast cancer cells. However, addition of CD134 (OX-40) or CD137 (4-1BB) signaling domain to a backbone of NKG2D-Z 1^{st} generation CAR did not exhibit higher cytotoxicity than the cytotoxicity of NKG2D-Z CAR (1^{st} generation). Cancer cell killing by NK-92 MI cells including NKG2D-28Z CAR (2^{nd} generation) was superior to the positive control, NK-92 MI cells expressing NKG2D-Z CAR (1^{st} generation) (FIG. 5B).

Increase of Tumor Killing Effect of NK92MI Cells Expressing NKG2D Receptor, which Contains CD28 Intracellular Domain, Against MCF7 Breast Cancer Cell Line

3^{rd} generation CAR is known to increase antitumor activity. In physiological T cell response, the optimal lymphocyte activation needs one or more cofactor receptors combined with a co-stimulatory molecule such as CD28. Among the cofactor receptors, the most important receptors are CD137 (4-1BB) and OX40 (CD134) which are members of tumor necrosis factor (TNFR).

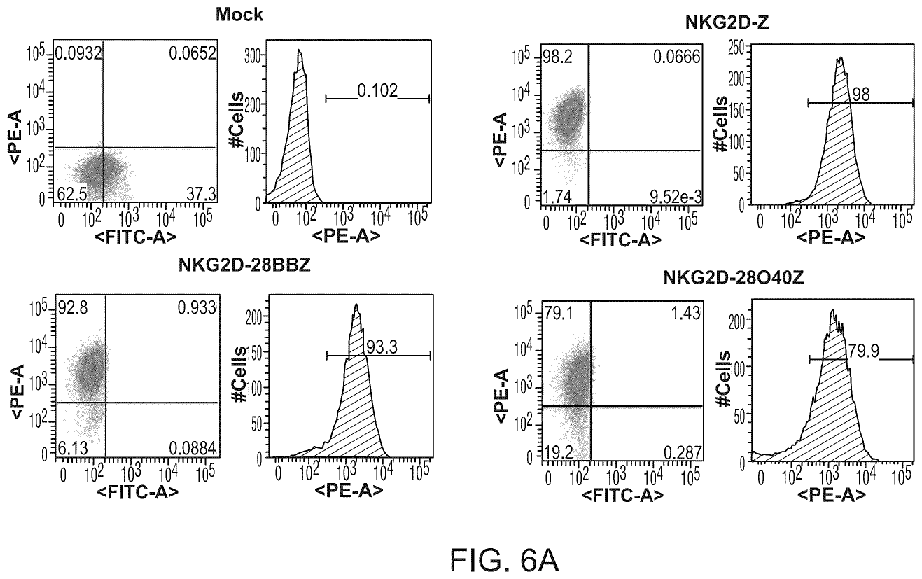

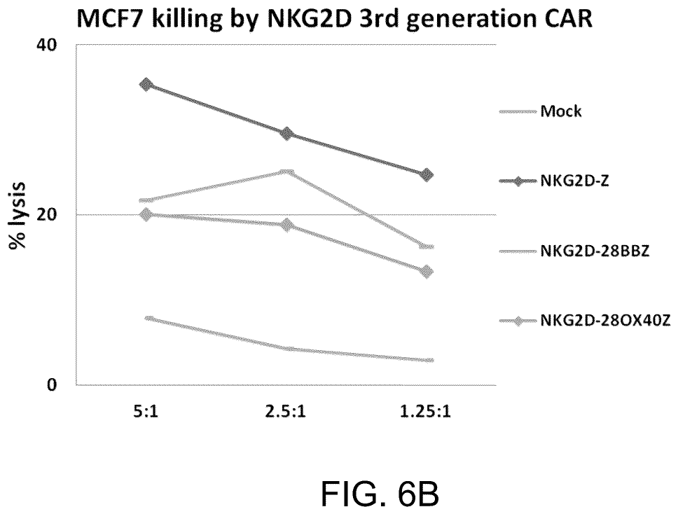

In order to investigate effects of different cofactors in NK-92 MI cells expressing NKG2D CAR (3^{rd} generation), the present inventors have produced NKG2D-28OX40Z CAR (3^{rd} generation) and NKG2D-28BBZ CAR (3^{rd} generation). The NKG2D CAR (3^{rd} generation) constructs were expressed in NK92MI cells using a lentiviral vector. The transduced NK92MI cells efficiently expressed various NKG2D receptors containing 3^{rd} generation CAR (FIG. 6A). As compared to NK92MI cells transduced with a control vector, NK92MI cells expressing various NKG2D CARs (3^{rd} generation) have efficiently killed MCF7 cells in vitro. However, 3^{rd} generation NKG2D-28OX40Z CAR and NKG2D-28BBZ CAR, each of which includes CD134 or CD137 co-stimulatory signaling domain in a backbone of NKG2D-28Z CAR (2^{nd} generation), did not exhibit higher cytotoxicity than NKG2D-Z CAR (1^{st} generation) (FIG. 6B).

### Comparison of NKG2D-AAA-28(H)OX40LZ CAR (3^{rd} Generation) and Other Receptors

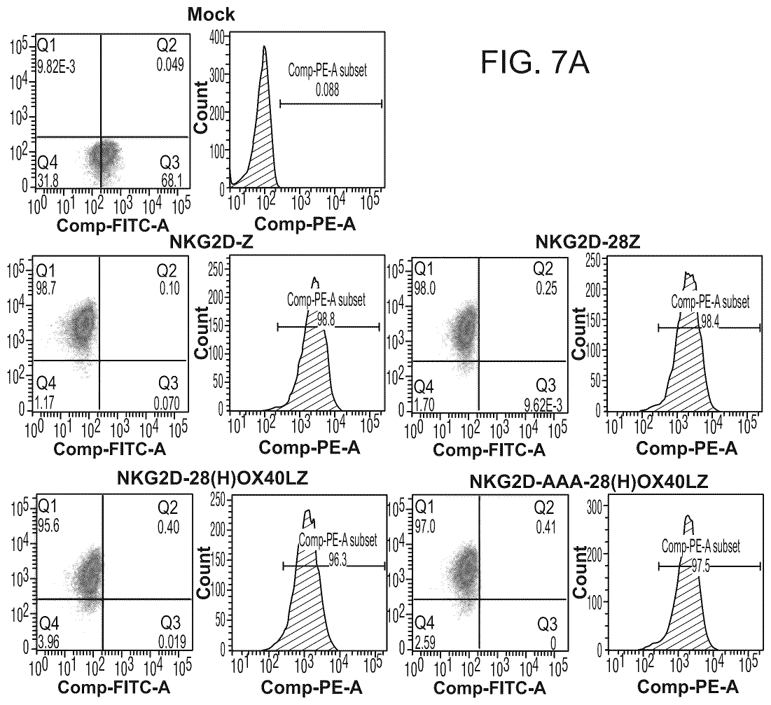

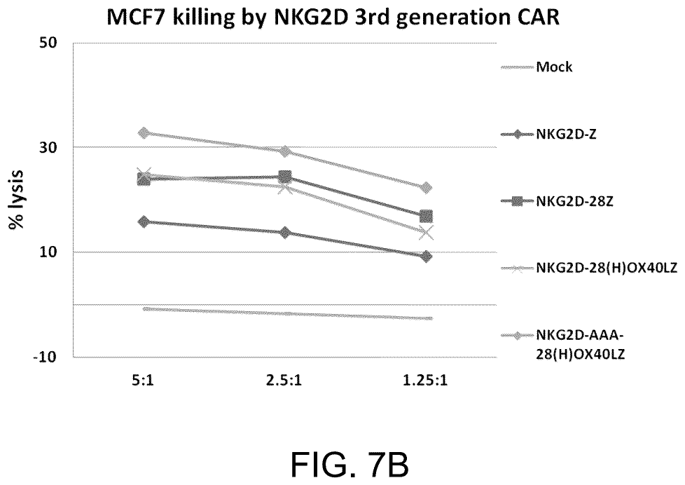

In order to assess CD252 (OX40 ligand) co-stimulatory signaling domain effects, the signaling domain of the OX40 ligand was fused to the backbone of NKG2D-28Z CAR (2^{nd} generation), so as to build NKG2D-28(H)OX40LZ CAR (3^{rd} generation). In the tumor killing assay, tumor specific cytotoxicity to MCF7 cells, which was induced by NKG2D-28(H)OX40LZ CAR (3^{rd} generation), is greatly enhanced compared to NKG2D-Z CAR (1^{st} generation), however, is substantially similar to NKG2D-28Z CAR (2^{nd} generation) (FIG. 7B).

For optimal CAR T-cell recognition, it was determined that, not only tumor antigen recognition receptor itself, but also hinge and linker sequences between the antigen-specific receptor and the cell membrane are also important. In the present experiment, additional effects of the spacer to the function of NKG2D-28(H)OX40LZ CAR (3^{rd} generation) were analyzed. The present inventors have introduced an AAA (triple alanine) linker between the NKG2D extracellular domain and CD28 hinge. NKG2D-AAA-28(H)OX40LZ CAR (3^{rd} generation) containing AAA linker introduced therein was expressed in NK92MI cells using a lentiviral vector (FIG. 7A). In the present experiment, MCF7 cell killing by NK-92 MI cells expressing NKG2D-AAA-28(H)OX40LZ CAR (3^{rd} generation) was compared with MCF7 cell killing by NK-92 MI cells expressing NKG2D-Z CAR (1^{st} generation), NKG2D-28Z CAR (2^{nd} generation) or NKG2D-28(H)OX40LZ CAR (3^{rd} generation). In particular, introduction of the AAA linker between NKG2D extracellular domain and CD28 hinge of NKG2D-28(H)OX40LZ CAR (3^{rd} generation) has resulted in better target cell killing than parental NKG2D-28(H)OX40LZ CAR (3^{rd} generation) without AAA, NKG2D-Z CAR (1^{st} generation) and/or NKG2D-28Z CAR (2^{nd} generation) (FIG. 7B).

### Increase of Tumor Killing Effect of NK92MI Cells Expressing NKG2D-AAA-28(H)OX40LZ CAR (3^{rd} Generation) Against the Lung Cancer Cell Line

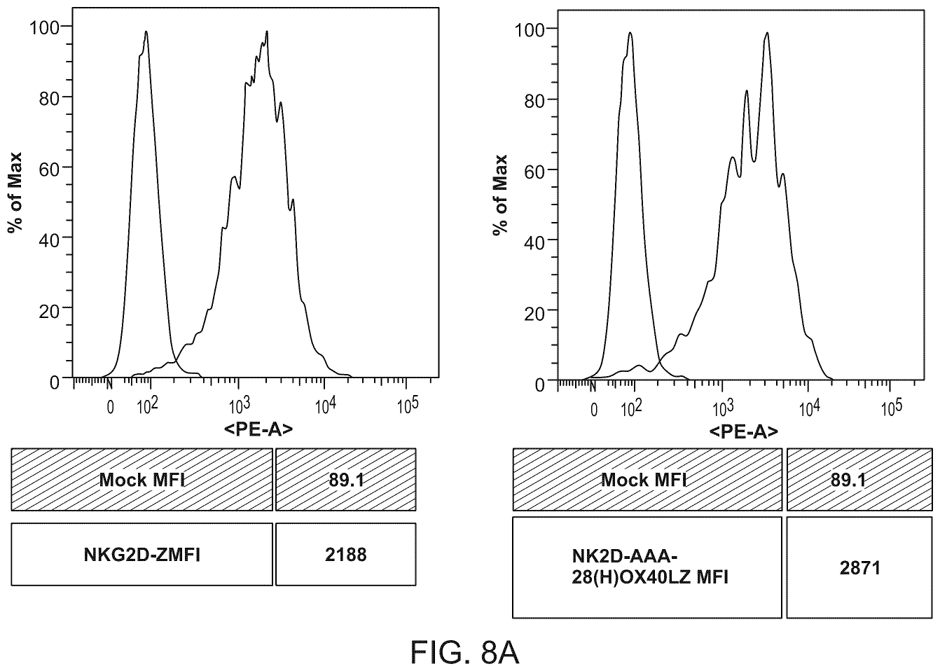

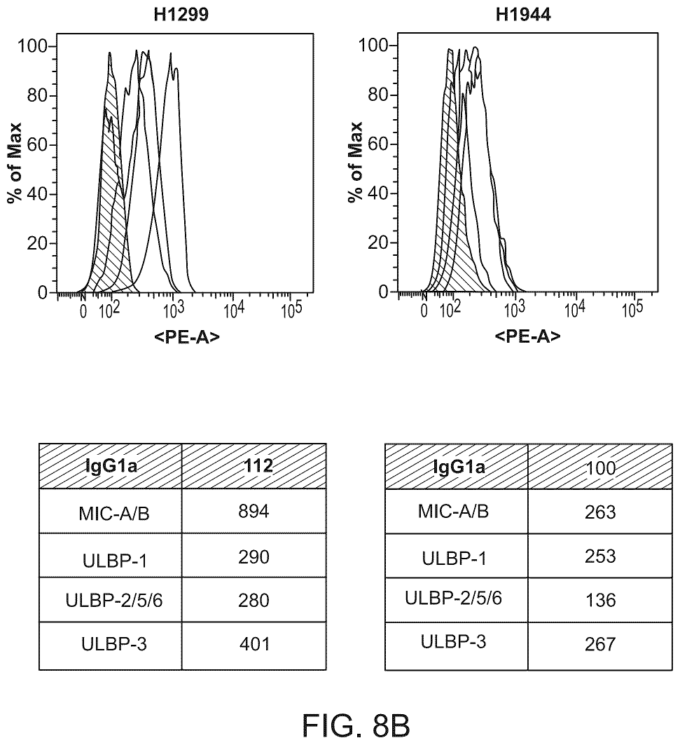

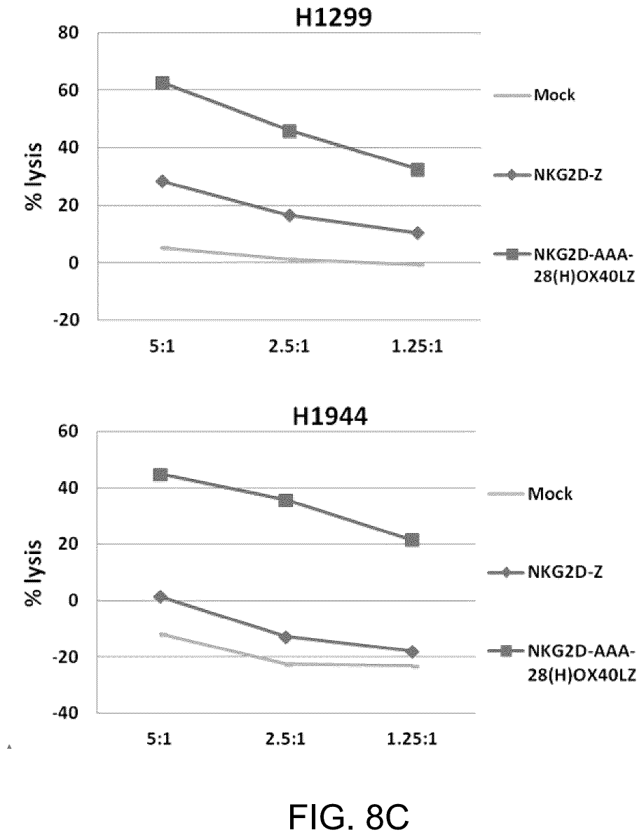

In order to assess whether NK92MI cells transduced with NKG2D-AAA-28(H)OX40LZ CAR (3^{rd} generation) can recognize NKG2D ligand in the lung cancer cells, NKG2D ligand-positive tumor cells (H1299 and H1944) were used as a target of NK92MI cells including NKG2D-AAA-28(H)OX40L CAR (3^{rd} generation). The transduced NK92MI cells efficiently expressed NKG2D-Z CAR (1^{st} generation) or NKG2D-AAA-28(H)OX40L CAR (3^{rd} generation) (FIG. 8A). Next, the NKG2D ligand expression in H1299 and H1944 cells was assessed. The present inventors have identified that the NKG2D ligand expression was detected in H1299 and H1944 cells by a flow cytometric analysis (FIG. 8B). As shown in FIG. 8C, NK92MI cells including NKG2D-AAA-28(H)OX40L CAR (3^{rd} generation) could more efficiently killed the target cells expressing NKG2D ligand in vitro than the NK92MI cells as a control group and NK92MI cells expressing NKG2D-Z CAR (1^{st} generation). Furthermore, specificity of target cell killing was obviously confirmed because H1299 and H1944 cells are not killed by NKG2D non-expressing NK92MI cells transduced by a control empty vector.

1. A chimeric antigen receptor, comprising:
an intracellular signaling domain which includes the whole or a portion of OX40 ligand (CD252).

2. The chimeric antigen receptor according to claim 1, further comprising:
a transmembrane domain linked to the intracellular signaling domain;
a spacer domain linked to the transmembrane domain; and
an extracellular domain linked to the spacer domain.

3. The chimeric antigen receptor according to claim 2, further comprising a signal sequence linked to the extracellular domain.

4. (canceled)

5. The chimeric antigen receptor according to claim 2, wherein the extracellular domain includes the whole or a portion of any one selected from the group consisting of an antigen-binding fragment of an antibody, a Fc receptor, a natural cytotoxicity receptor, NKG2D, 2B4 and DNAM-1.

6. (canceled)

7. The chimeric antigen receptor according to claim 5, wherein the antigen-binding fragment is a single-chain variable fragment (ScFv).

8.-10. (canceled)

11. The chimeric antigen receptor according to claim 2, wherein the spacer domain includes the whole or a portion of any one selected from the group consisting of CD8a and CD28.

12. The chimeric antigen receptor according to claim 2, wherein the transmembrane domain includes the whole or a portion of any one selected from the group consisting of CD8a and CD28.

13. The chimeric antigen receptor according to claim 1, wherein the intracellular signaling domain further includes the whole or a portion of CD3-zeta.

14. (canceled)

15. A chimeric antigen receptor, comprising intracellular signaling domains which comprise:
a first intracellular signaling domain including the whole or a portion of any one selected from the group consisting of CD28 and 4-1BB;
a second intracellular signaling domain including the whole or a portion of OX40 ligand; and
a third intracellular signaling domain including the whole or a portion of CD3-zeta,
wherein the first, second and third intracellular signaling domains are arranged in order from a cell membrane toward an inside of the cell.

16. The chimeric antigen receptor according to claim 15, further comprising:
a transmembrane domain linked to the intracellular signaling domain;
a spacer domain linked to the transmembrane domain;
and an extracellular domain linked to the spacer domain.

17. The chimeric antigen receptor according to claim 16, further comprising a signal sequence linked to the extracellular domain.

18. (canceled)

19. The chimeric antigen receptor according to claim 16, wherein the extracellular domain includes the whole or a portion of any one selected from the group consisting of an antigen-binding fragment of an antibody, a Fc receptor, a natural cytotoxicity receptor, NKG2D, 2B4 and DNAM-1.

20. The chimeric antigen receptor according to claim 16, wherein the spacer domain includes the whole or a portion of any one selected from the group consisting of CD8α and CD28.

21. The chimeric antigen receptor according to claim 16, wherein the transmembrane domain includes the whole or a portion of any one selected from the group consisting of CD8α and CD28.

22. The chimeric antigen receptor according to claim 15, wherein the first intracellular signaling domain includes the whole or a portion of CD28;
the second intracellular signaling domain includes the whole or a portion of the OX40 ligand; and
the third intracellular signaling domain includes the whole or a portion of CD3-zeta.

23. An immune cell expressing the chimeric antigen receptor according to claim 1.

24. The immune cell according to claim 23, wherein the immune cell is a natural killer cell (NK cell).

25. A pharmaceutical composition for treatment of tumor, comprising the immune cell according to claim 23 as an active ingredient.

26. (canceled)

27. A nucleic acid sequence encoding the chimeric antigen receptor according to claim 1.

28. (canceled)

29. (canceled)

30. A method for treatment of tumor, comprising administering the immune cell according to claim 23 to a subject.


SEQUENCE LISTING (93 sequences)

SEQ ID NO: 1 - DNA, 618 bp, synthetic construct
  source 1..618: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctggg

SEQ ID NO: 2 - protein, 206 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPG

SEQ ID NO: 3 - DNA, 618 bp, synthetic construct
  source 1..618: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctggg

SEQ ID NO: 4 - DNA, 618 bp, synthetic construct
  source 1..618: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggg

SEQ ID NO: 5 - protein, 206 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPG

SEQ ID NO: 6 - DNA, 216 bp, synthetic construct
  source 1..216: mol_type = other DNA
gcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 7 - DNA, 216 bp, synthetic construct
  source 1..216: mol_type = other DNA
gccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtctctggtcatcactctgtattgt

SEQ ID NO: 8 - protein, 72 aa, synthetic construct
AKPTTTPAPRPPTPAPTIASQPLSLRPEACRPAAGGAVHTRGLDFACDIYIWAPLAGTCGVLLLSLVITLYC

SEQ ID NO: 9 - DNA, 321 bp, synthetic construct
  source 1..321: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 10 - DNA, 321 bp, synthetic construct
  source 1..321: mol_type = other DNA
atcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagt

SEQ ID NO: 11 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 12 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 13 - DNA, 336 bp, synthetic construct
  source 1..336: mol_type = other DNA
cgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 14 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 66 bp, synthetic construct
  source 1..66: mol_type = other DNA
gaaagggtccaacccctggaagagaatgtgggaaatgcagccaggccaagattcgagaggaacaag

SEQ ID NO: 16 - DNA, 66 bp, synthetic construct
  source 1..66: mol_type = other DNA
gaaagagtgcagcccctggaagagaatgtcgggaatgccgctcgcccaagatttgaaaggaacaaa

SEQ ID NO: 17 - protein, 22 aa, synthetic construct
ERVQPLEENVGNAARPRFERNK

SEQ ID NO: 18 - DNA, 108 bp, synthetic construct
  source 1..108: mol_type = other DNA
agggaccagaggctgccccccgatgcccacaagccccctgggggaggcagtttccggacccccatccaagaggagcaggccgacgcccactccaccctggccaagatc

SEQ ID NO: 19 - DNA, 108 bp, synthetic construct
  source 1..108: mol_type = other DNA
cgggaccagagactgccacctgatgcacacaagccaccaggaggaggcagcttcaggacccccatccaggaggaacaggccgacgctcattccacactggccaaaatt

SEQ ID NO: 20 - protein, 36 aa, synthetic construct
RDQRLPPDAHKPPGGGSFRTPIQEEQADAHSTLAKI

SEQ ID NO: 21 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 22 - DNA, 126 bp, synthetic construct
  source 1..126: mol_type = other DNA
aagcggggaagaaagaaactgctgtacatcttcaaacagccctttatgaggcctgtgcagaccacacaggaggaagacggctgctcctgccggttccccgaggaagaggaaggcgggtgcgagctg

SEQ ID NO: 23 - protein, 42 aa, synthetic construct
KRGRKKLLYIFKQPFMRPVQTTQEEDGCSCRFPEEEEGGCEL

SEQ ID NO: 24 - DNA, 1173 bp, synthetic construct
  source 1..1173: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtctctggtcattactctgtattgtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 25 - protein, 390 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 1299 bp, synthetic construct
  source 1..1299: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtctctggtcattactctgtattgtaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 27 - protein, 432 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 1281 bp, synthetic construct
  source 1..1281: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtccctggtcatcactctgtattgtcgggaccagagactgccacctgatgcacacaagccaccaggaggaggcagcttcaggacccccatccaggaggaacaggccgacgctcattccacactggccaaaattcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 29 - protein, 426 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDIYIWAPLAGTCGVLLLSLVITLYCRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 1239 bp, synthetic construct
  source 1..1239: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtctctggtcattactctgtattgtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 31 - protein, 412 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDIYIWAPLAGTCGVLLLSLVITLYCERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 1305 bp, synthetic construct
  source 1..1305: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 33 - protein, 434 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 1278 bp, synthetic construct
  source 1..1278: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctgggatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 35 - protein, 425 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 1407 bp, synthetic construct
  source 1..1407: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtctctggtcattactctgtattgtaagcggggaagaaagaaactgctgtacatcttcaaacagccctttatgaggcctgtgcagaccacacaggaggaagacggctgctcctgccggttccccgaggaagaggaaggcgggtgcgagctgcgggaccagagactgccacctgatgcacacaagccaccaggaggaggcagcttcaggacccccatccaggaggaacaggccgacgctcattccacactggccaaaattcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 37 - protein, 468 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 1407 bp, synthetic construct
  source 1..1407: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtccctggtcatcactctgtattgtcgggaccagagactgccacctgatgcacacaagccaccaggaggaggcagcttcaggacccccatccaggaggaacaggccgacgctcattccacactggccaaaattaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 39 - protein, 468 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDIYIWAPLAGTCGVLLLSLVITLYCRDQRLPPDAHKPPGGGSFRTPIQEEQADAHSTLAKIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 1431 bp, synthetic construct
  source 1..1431: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 41 - protein, 476 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 1413 bp, synthetic construct
  source 1..1413: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgggaccagagactgccacctgatgcacacaagccaccaggaggaggcagcttcaggacccccatccaggaggaacaggccgacgctcattccacactggccaaaattcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 43 - protein, 470 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 1371 bp, synthetic construct
  source 1..1371: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 45 - protein, 456 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 1404 bp, synthetic construct
  source 1..1404: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctgggatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 47 - protein, 467 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 1386 bp, synthetic construct
  source 1..1386: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctgggatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgggaccagagactgccacctgatgcacacaagccaccaggaggaggcagcttcaggacccccatccaggaggaacaggccgacgctcattccacactggccaaaattcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 49 - protein, 461 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - DNA, 1344 bp, synthetic construct
  source 1..1344: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctgggatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 51 - protein, 447 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 1365 bp, synthetic construct
  source 1..1365: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtctctggtcattactctgtattgtaagcggggaagaaagaaactgctgtacatcttcaaacagccctttatgaggcctgtgcagaccacacaggaggaagacggctgctcctgccggttccccgaggaagaggaaggcgggtgcgagctggaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 53 - protein, 454 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - DNA, 1239 bp, synthetic construct
  source 1..1239: mol_type = other DNA
atgtggcagctgctgctgcctaccgctctgctgctgctggtctccgctgggatgcgaaccgaggatctgcctaaagccgtggtcttcctggagcctcagtggtacagagtgctggagaaggactctgtgacactgaaatgccagggcgcttattcaccagaggataacagcactcagtggttccacaatgaatccctgatcagctcccaggcatctagttactttattgacgccgctacagtggacgattctggagagtatcgatgccagactaacctgagcaccctgtccgatcccgtgcagctggaagtccacatcggatggctgctgctccaggcaccaagatgggtcttcaaggaggaagaccccattcacctgcgctgtcatagctggaagaataccgctctgcataaagtgacatacctccagaacggaaagggccgaaaatacttccaccataattccgacttttatatccccaaggcaaccctgaaagatagtgggtcatatttttgtcgggggctggtgggaagtaaaaacgtctcaagcgagactgtgaatatcaccattacacagggcctggctgtcagcaccatctcctctttctttccccctggggccaaacctaccacaactcctgctccaagaccacccacacccgctcctactattgcatctcagccactgagtctgcgaccagaggcctgccggcccgccgccggcggggccgtgcataccaggggcctggacttcgcctgtgatatctacatttgggctccactggctgggacttgcggcgtgctgctgctgtctctggtcattactctgtattgtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgggaaagagtgcagcccctggaagagaatgtcgggaatgccgctcgcccaagatttgaaaggaacaaatga

SEQ ID NO: 55 - protein, 412 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGAKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRERVQPLEENVGNAARPRFERNK

SEQ ID NO: 56 - DNA, 63 bp, synthetic construct
  source 1..63: mol_type = other DNA
atggccttaccagtgaccgccttgctcctgccgctggccttgctgctccacgccgccaggccg

SEQ ID NO: 57 - DNA, 63 bp, synthetic construct
  source 1..63: mol_type = other DNA
atggctctgccagtgactgcactgctgctgccactggccctgctgctgcacgcagctcgacct

SEQ ID NO: 58 - protein, 21 aa, synthetic construct
MALPVTALLLPLALLLHAARP

SEQ ID NO: 59 - DNA, 405 bp, synthetic construct
  source 1..405: mol_type = other DNA
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 60 - DNA, 405 bp, synthetic construct
  source 1..405: mol_type = other DNA
ctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtg

SEQ ID NO: 61 - protein, 135 aa, synthetic construct
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 62 - DNA, 1023 bp, synthetic construct
  source 1..1023: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 63 - protein, 340 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - DNA, 1149 bp, synthetic construct
  source 1..1149: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 65 - protein, 382 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 1131 bp, synthetic construct
  source 1..1131: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtcgggaccagagactgccacctgatgcacacaagccaccaggaggaggcagcttcaggacccccatccaggaggaacaggccgacgctcattccacactggccaaaattcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 67 - protein, 376 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDIYIWAPLAGTCGVLLLSLVITLYCRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 1089 bp, synthetic construct
  source 1..1089: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgggaaagagtgcagcccctggaagagaatgtcgggaatgccgctcgcccaagatttgaaaggaacaaatga

SEQ ID NO: 69 - protein, 362 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRERVQPLEENVGNAARPRFERNK

SEQ ID NO: 70 - DNA, 1155 bp, synthetic construct
  source 1..1155: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 71 - protein, 384 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - DNA, 1128 bp, synthetic construct
  source 1..1128: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtgatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 73 - protein, 375 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1257 bp, synthetic construct
  source 1..1257: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtaagcggggaagaaagaaactgctgtacatcttcaaacagccctttatgaggcctgtgcagaccacacaggaggaagacggctgctcctgccggttccccgaggaagaggaaggcgggtgcgagctgcgggaccagagactgccacctgatgcacacaagccaccaggaggaggcagcttcaggacccccatccaggaggaacaggccgacgctcattccacactggccaaaattcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 75 - protein, 418 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - DNA, 1215 bp, synthetic construct
  source 1..1215: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtaagcggggaagaaagaaactgctgtacatcttcaaacagccctttatgaggcctgtgcagaccacacaggaggaagacggctgctcctgccggttccccgaggaagaggaaggcgggtgcgagctggaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 77 - protein, 404 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - DNA, 1257 bp, synthetic construct
  source 1..1257: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatatctacatttgggcccctctggctggaacatgtggcgtgctgctgctgtccctggtcattactctgtattgtcgggaccagagactgccacctgatgcacacaagccaccaggaggaggcagcttcaggacccccatccaggaggaacaggccgacgctcattccacactggccaaaattaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 79 - protein, 418 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDIYIWAPLAGTCGVLLLSLVITLYCRDQRLPPDAHKPPGGGSFRTPIQEEQADAHSTLAKIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - DNA, 1281 bp, synthetic construct
  source 1..1281: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 81 - protein, 426 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 1263 bp, synthetic construct
  source 1..1263: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgggaccagagactgccacctgatgcacacaagccaccaggaggaggcagcttcaggacccccatccaggaggaacaggccgacgctcattccacactggccaaaattcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 83 - protein, 420 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - DNA, 1221 bp, synthetic construct
  source 1..1221: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 85 - protein, 406 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - DNA, 1254 bp, synthetic construct
  source 1..1254: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtgatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtaagcggggaagaaagaaactgctgtacatcttcaaacagccctttatgaggcctgtgcagaccacacaggaggaagacggctgctcctgccggttccccgaggaagaggaaggcgggtgcgagctgcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 87 - protein, 417 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - DNA, 1236 bp, synthetic construct
  source 1..1236: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtgatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtcgggaccagagactgccacctgatgcacacaagccaccaggaggaggcagcttcaggacccccatccaggaggaacaggccgacgctcattccacactggccaaaattcgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 89 - protein, 411 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - DNA, 1194 bp, synthetic construct
  source 1..1194: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtgatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 91 - protein, 397 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - DNA, 1203 bp, synthetic construct
  source 1..1203: mol_type = other DNA
atggctctgccagtgactgcactgctgctgccactggccctgctgctgcacgcagctcgacctctgttcaaccaggaggtccagatcccactgaccgaaagttactgcggaccatgtcccaagaactggatctgctacaagaacaactgttaccagttctttgacgagtctaagaactggtatgaatctcaggccagttgcatgtcacagaatgcttcactgctgaaggtgtacagcaaagaggaccaggatctgctgaagctggtgaaatcctatcactggatgggcctggtccatatcccaaccaacgggtcttggcagtgggaggacggaagcattctgtcccccaatctgctgacaatcattgaaatgcagaagggcgattgtgctctgtacgcaagctccttcaaagggtatatcgagaactgctccacccccaatacatacatttgtatgcagaggacagtggccgccgctatcgaggtcatgtacccccctccatatctggacaacgaaaagtccaatggaactatcattcacgtgaagggcaaacatctgtgcccatctcccctgttccctgggccaagtaaacctttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggtga

SEQ ID NO: 93 - protein, 400 aa, synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR